(19) United States
(12) Patent Application Publication
(10) Pub. No.: US 2026/0318805 A1
(43) Pub. Date: Oct. 15, 2026

(54) PHOTONIC GAS SENSOR

(71) Applicant: VTEC Lasers & Sensors Ltd, 5616LZ Eindhoven (NL)

(72) Inventor: Jan Mink, Geldrop (NL)

(73) Assignee: VTEC Lasers & Sensors Ltd, 5616LZ Eindhoven (NL)

(21) Appl. No.: 19/184,933

(22) Filed: Apr. 21, 2025

Related U.S. Application Data
(60) Provisional application No. 63/636,449, filed on Apr. 19, 2024.
(60) Provisional application No. 63/683,353, filed on Aug. 15, 2024.

(51) Int. Cl. G01N 15/075; G01N 15/06; G01N 21/63; G01N 33/00

(52) U.S. Cl. CPC G01N 15/075; G01N 21/636; G01N 33/0054; G01N 2015/0662

(57) ABSTRACT

Method and apparatuses are provided for determining gas sample concentration by exciting molecules of a gas sample with an excitation laser and applying a probe modulation signal to a probe laser. The probe laser's output is split into a reference beam and a test beam, with the test beam passed through the sample reservoir, then the reference beam and test beam are recombined to create a signal representing relative phase shift variations between the test beam and the reference beam. This signal is detected and processed to determine gas sample concentration data.

This application claims the benefit of U.S. provisional patent application Ser. No. 63/636,449, filed on Apr. 19, 2024, and the benefit of U.S. provisional patent application Ser. No. 63/683,353, filed on Aug. 15, 2024, each of which is incorporated herein by reference in its entirety.

### BACKGROUND

Compact, rugged, and sensitive laser-based trace gas sensors are in high demand for scientific and commercial applications. As these applications expand, the demand is expected to rise. One area of particular interest and potential expansion is the need for measurement technologies that may be needed to deploy solutions for the reduction of carbon dioxide, methane, and other greenhouse gases in the atmosphere.

For example, in 2022 approximately 11 gigatons (Gt) of CO₂ were emitted globally. Industrial processes may gradually become more electrified, with a goal of reducing the carbon footprint to approximately zero in 2050. However, low-carbon fuels may still be required in a future, decarbonized energy landscape, as not all industrial processes qualify for electrification. Greenhouse gases may remain the expected byproducts of low-carbon fuel consumption and may need to be rigorously measured and regulated or mitigated. More information is available form the Institute for Sustainable Process Technology at https://ispt.eu.

Green hydrogen is a zero-carbon fuel and may be an important energy carrier to accommodate CO₂ reduction. However, due to its chemical properties, it has a relatively low energy density, which makes it less attractive for transport, storage, and utilization. It also has a low ignition energy, increasing explosion risks. Alternatively, hydrogen may be stored as part of an ammonia compound, which may help resolve these issues. Ammonia as a hydrogen carrier may be converted back to hydrogen again without the carbon byproducts created when using other conventional hydrogen carriers. Safe, widespread use of green hydrogen and ammonia-stored hydrogen may also necessitate economically viable commercial solutions for gas measurement that may be quickly and easily deployed in the field.

Direct absorption (DA) spectroscopy and photothermal spectroscopy (PTS) are long-standing techniques for determining the contents and concentration of gas samples, and their accuracy may be improved upon by employing wavelength modulation spectroscopy (WMS) techniques. WMS techniques may provide a number of noise-rejection benefits but may do so at the cost of increased complexity of the signal processing needed to interpret the output results, as well as the more complex capabilities needed for the excitation laser, in addition to the higher power needs already imposed for the excitation laser to adequately heat or excite the molecules of the sample under test.

Complex excitation lasers may be large and expensive, reducing the effectiveness of these techniques in applications where small, inexpensive sensing capabilities are needed. In addition, to ensure high sensitivities, laser spectroscopic sensors often use extended interaction paths (e.g., multi-pass cells), which may significantly increase their size, weight, and susceptibility to misalignment, further reducing their efficacy in numerous applications under real-world conditions.

There is, therefore, a need for trace gas sensors that may reliably detect the presence of and measure the concentration of a range of gases, under various real-world and potentially harsh conditions, while remaining small enough and inexpensive enough for a broad range of commercial implementations.

In various embodiments, a method is used to analyze the concentration of a gas sample by exciting the gas molecules with an excitation laser and applying a probe modulation signal to a probe laser. The probe laser is split into a test beam and a reference beam. The excitation laser and test beam are passed through the gas sample, then the test beam and reference beam are recombined to create a signal representing relative phase shift variations between the test beam and the reference beam.

The recombined signal is detected using a photodetector, and the detected signal is received by a transimpedance amplifier to generate a voltage signal. This voltage signal is then received by a lock-in amplifier along with the probe modulation signal, which generates a lock-in amplifier output signal comparing the voltage signal and the probe modulation signal.

The lock-in amplifier output signal is then converted to a digital output signal using an analog to digital converter, and the digital output signal is processed by a signal processing module to determine the gas sample concentration data.

In some embodiments, the method involves exciting the gas molecules with an excitation laser, applying an excitation modulation signal to the excitation laser, or using a different detection method, such as a beat node detector.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates conventional WMS gas sensor operation 100.

FIG. 2 illustrates a conventional WMS gas sensor 200.

FIG. 3A and FIG. 3B illustrate a 2f WMS signal 300.

FIG. 4 illustrates a photonic gas sensor 400 in accordance with one embodiment.

FIG. 5 illustrates a signal acquisition 500 in accordance with one embodiment.

FIG. 6 illustrates noise cancellation and accuracy improvement 600 in accordance with one embodiment.

FIG. 7 illustrates probe signal tuning 700 in accordance with one embodiment.

FIG. 8 illustrates excitation signal tuning 800 in accordance with one embodiment.

FIG. 9 illustrates a photonic gas sensor 900 in accordance with one embodiment.

FIG. 10 illustrates a photonic gas sensor 1000 in accordance with one embodiment.

FIG. 11 illustrates a block signal 1100 in accordance with one embodiment.

FIG. 12 illustrates photonic gas sensor 1200 in accordance with one embodiment.

FIG. 13 illustrates a chip-based photonic gas sensor 1300 in accordance with one embodiment.

FIG. 14 illustrates a chip-based photonic gas sensor 1400 in accordance with one embodiment.

FIG. 15 illustrates a chip-based photonic gas sensor 1500 in accordance with one embodiment.

FIG. 16 illustrates a chip-based photonic gas sensor 1600 in accordance with one embodiment.

FIG. 17 illustrates a chip-based pump laser 1700 in accordance with one embodiment.

FIG. 18 illustrates a chip-based photonic gas sensor 1800 in accordance with one embodiment.

FIG. 19 illustrates a photonic gas sensor for harsh environments 1900 in accordance with one embodiment.

FIG. 20 illustrates a photonic gas sensor for harsh environments 2000 in accordance with one embodiment.

FIG. 21 illustrates a discrete beat node ammonia sensor 2100 in accordance with one embodiment.

FIG. 22A and FIG. 22B illustrate a transmitter optical sub-assembly (TOSA 2200) in accordance with one embodiment.

FIG. 23A and FIG. 23B illustrate a receiver optical sub-assembly (ROSA 2300) in accordance with one embodiment.

FIG. 24A-FIG. 24D illustrate a real-time calibration configuration with a reference cell 2400 in accordance with one embodiment.

FIG. 25A-FIG. 25G illustrate a photonic gas sensor with configurable add-ons 2500 in accordance with one embodiment.

FIG. 26 illustrates an exemplary solution 2600 in accordance with one embodiment.

FIG. 27 illustrates a test sample reservoir 2700 in accordance with one embodiment.

FIG. 28 illustrates sensor output versus ammonia sample concentration data 2800 in accordance with one embodiment.

FIG. 29 illustrates an ammonia setup 2900 in accordance with one embodiment.

FIG. 30 illustrates an exemplary assembly integration 3000 in accordance with one embodiment.

FIG. 31A-FIG. 31D illustrate a transceiver optics concept 3100 in accordance with one embodiment.

FIG. 32A illustrates an ammonia sensor optics concept 3200a in accordance with one embodiment.

FIG. 32B illustrates an ammonia sensor with a packaged Mach-Zehnder interferometer (MZI) 3200b in accordance with one embodiment.

FIG. 33 illustrates an ammonia sensor optics concept with a multi-pass cell configuration 3300 in accordance with one embodiment.

FIG. 34 illustrates an ammonia sensor optics concept 3500 in accordance with one embodiment.

FIG. 35 illustrates an ammonia sensor optics concept 3500 in accordance with one embodiment.

FIG. 36 illustrates ammonia sensor optics concept with offset compensation 3600 in accordance with one embodiment.

FIG. 37 illustrates multi-gas sensor optics concept with offset compensation 3700 in accordance with one embodiment.

FIG. 38 illustrates ammonia sensor optics concept with compensation of external environment influences 3800 in accordance with one embodiment.

FIG. 39 illustrates ammonia sensor optics concept with compensation for external environment influences 3900 in accordance with one embodiment.

FIG. 40 illustrates a transmission spectrum of an imbalanced MZI 4000 in accordance with one embodiment.

FIG. 41 illustrates a maximum lock-in amplifier output for ammonia at 10 ppm 4100 in accordance with one embodiment.

FIG. 42 illustrates a lock-in amplifier output for 100 ppm ammonia 4200 in accordance with one embodiment.

### DETAILED DESCRIPTION

Herein, disclosed is a miniaturized photothermal gas sensor, where the gas sample is measured inside the resonator of a monolithic microchip solid-state laser operating at 1310 nm. The photothermal-induced gas refractive index variations are directly translated to a solid-state laser frequency shift, which is detected as a beat node modulation in a heterodyne detection scheme or may be detectable as a phase shift across the legs of a Mach-Zehnder interferometer (MZI).

The system provides high sensitivity to refractive index changes at the level of ~1.1×10-\hat{ }12 within ultra-short intra-cavity interaction path-length of 1.5 mm, which may support trace-gas measurements in a sensing volume as small as 4 μl.

In a proof-of-concept experiment using dry carbon dioxide as a test sample, the sensor reached a minimum detection limit of 350 parts per billion by volume (ppbv) for a 100 s averaging time and a normalized noise equivalent absorption (NNEA) of 4.1×10-\hat{ }8 [W cm-\hat{ }1 Hz-\hat{ }½].

The disclosed sensor may find application in the fields of agriculture, especially livestock management, fruit transport, ammonia combustion, ammonia transport for hydrogen fuel, gas detection in harsh environments (using transparent high-strength glass), accurate detections in high concentrations of gas, areas where automatic adjustment for a range of gases is needed, identification of a gas using photothermal spectroscopy (PTS) while scanning a wavelength range, and identification of multiple gases using local machine learning algorithms while scanning a wavelength range using trained data sets with gas absorption spectra.

In one embodiment, the disclosed solution may perform a process including:
exciting, using an excitation laser, molecules of a gas sample in a sample reservoir;
applying, from a lock-in sine wave signal generator, a probe modulation signal to a probe laser;
splitting, by a Mach-Zehnder interferometer, the probe laser output into a reference beam and a test beam;
passing the test beam through the sample reservoir;
recombining the test beam and the reference beam to create a recombined signal representing relative phase shift variations between the test beam and the reference beam;
detecting the recombined signal using a photodetector;
receiving, by a transimpedance amplifier, the detected recombined signal to generate a voltage signal carrying data from the photodetector;
receiving, by a lock-in amplifier, the voltage signal from the transimpedance amplifier and the probe modulation signal from the lock-in sine wave signal generator to generate a lock-in amplifier output signal comparing the voltage signal and the probe modulation signal;
converting, by an analog to digital converter, the lock-in amplifier output signal to a digital output signal; and
processing, by a signal processing module, the digital output signal to determine gas sample concentration data.

In one embodiment, the disclosed solution may perform a process including:
exciting, using an excitation laser, molecules of a gas sample in a sample reservoir;
applying, from a lock-in sine wave signal generator, a probe modulation signal to a probe laser;
directing, by an optical splitter, the probe laser output into a measuring Mach-Zehnder interferometer and into an offset Mach-Zehnder interferometer splitting, by a measuring Mach-Zehnder interferometer, the probe laser output into a reference beam and a test beam;
passing the test beam through the sample reservoir;
recombining the test beam and the reference beam to create a recombined signal representing relative phase shift variations between the test beam and the reference beam;
detecting the recombined signal using a photodetector;
receiving, by a transimpedance amplifier, the detected recombined signal to generate a voltage signal carrying data from the photodetector;
splitting, by a phase reference Mach-Zehnder interferometer, the probe laser output into a reference beam and a test beam;
passing the test beam not through the sample reservoir;
recombining the test beam and the reference beam to create a recombined signal representing relative phase shift variations between the test beam and the reference beam;
detecting the recombined signal using a photodetector;
receiving, by a transimpedance amplifier, the detected recombined signal to generate a voltage signal carrying data from the photodetector;
subtracting the measuring Mach-Zehnder interferometer detector signal and the phase reference Mach-Zehnder interferometer detector signal in a differential amplifier;
receiving, by a lock-in amplifier, the voltage signal from the transimpedance amplifier and the probe modulation signal from the lock-in sine wave signal generator to generate a lock-in amplifier output signal comparing the voltage signal and the probe modulation signal;
converting, by an analog to digital converter, the lock-in amplifier output signal to a digital output signal; and
processing, by a signal processing module, the digital output signal to determine gas sample concentration data.

In one embodiment, the disclosed solution may perform a process including:
exciting, using an excitation laser, molecules of a gas sample in a sample reservoir;
applying, from a thermal wavelength controlling signal for the excitation laser;
applying, from a lock-in sine wave signal generator, a probe modulation signal to an excitation laser;
splitting, by a Mach-Zehnder interferometer, the probe laser output into a reference beam and a test beam;
passing the test beam through the sample reservoir;
recombining the test beam and the reference beam to create a recombined signal representing relative phase shift variations between the test beam and the reference beam;
detecting the recombined signal using a photodetector;
receiving, by a transimpedance amplifier, the detected recombined signal to generate a voltage signal carrying data from the photodetector;
receiving, by a lock-in amplifier, the voltage signal from the transimpedance amplifier and the probe modulation signal from the lock-in sine wave signal generator to generate a lock-in amplifier output signal comparing the voltage signal and the probe modulation signal;
converting, by an analog to digital converter, the lock-in amplifier output signal to a digital output signal; and
processing, by a signal processing module, the digital output signal to determine gas sample concentration data.

In one embodiment, the disclosed solution may include a gas sensor comprising:
a transmitting optical subassembly where the transmitting optical assembly contains:
   a semiconductor excitation laser, a monitor photodiode receiving the back side light from the excitation laser;
   a thermoelectric cooler (TEC) and thermistor connected to the excitation laser, a collimating lens collimating the excitation laser beam;
   a semiconductor probe laser, a monitor photodiode receiving the back side light from the excitation laser;
   a TEC and thermistor connected to the probe laser, a collimating lens collimating the excitation laser beam; and
   a beam combining device (Z-block) creating a Mach Zehnder with one combined excitation laser and probe laser beam and one leg containing the probe laser beam;
a sampling area, where beams pass through the sampling area where a gas or gas mixture is located;
a receiving optical sub assembly, where the two beams enter a beam separating device (Z-block), including:
   a photodiode receiving the light from the excitation laser; and
   a photodiode receiving the light from the two beams of the beam separating device; and
an electronics assembly comprising:
   a laser and TEC driver for controlling the excitation and probe laser output power and laser temperature;
   a signal generator for the lock-in amplifier for the excitation laser and the probe laser; and
   transimpedance amplifiers for the excitation laser detector signal and the Mach Zehnder detector signal; and
   a microcontroller for:
      controlling and monitoring the laser power and laser temperature of the probe and excitation lasers;
      controlling the wavelength of the probe laser by temperature;
      controlling the wavelength of the excitation laser by temperature;
      analog to digital converter (ADC) signal output processing; and
      communication of measured results, sensor status information, accepting control signals for adapting measurements process.

In one embodiment, the disclosed solution may include a photonics chip comprising:
at least one laser;
a splitter for creating the legs for a Mach-Zehnder interferometer (MZI);
in each leg, a phase matching section for compensation of phase changes caused by the probe laser wavelength change; and
at the rear side (not the MZI side) of the laser, a waveguide monitoring photodiode.

The photonics chip may also include at least one of:
a signal detection (planar or waveguide) photodiode for the MZI lock-in signal;
an excitation laser;
a wavelength locker and detecting photodiode for probe or excitation laser for wavelength control; and
a second splitter for creating the legs of a second Mach Zehnder interferometer to compensate for phase shifts from the probe laser wavelength.

The disclosed solution may also incorporate any of the following features:
a multi-pass cell;
heating of windows;
a reference cell;
an isolator for both lasers;
a protection insert;
a wavelength locker for both legs; and
an insert for adding another excitation wavelength and notch filter.

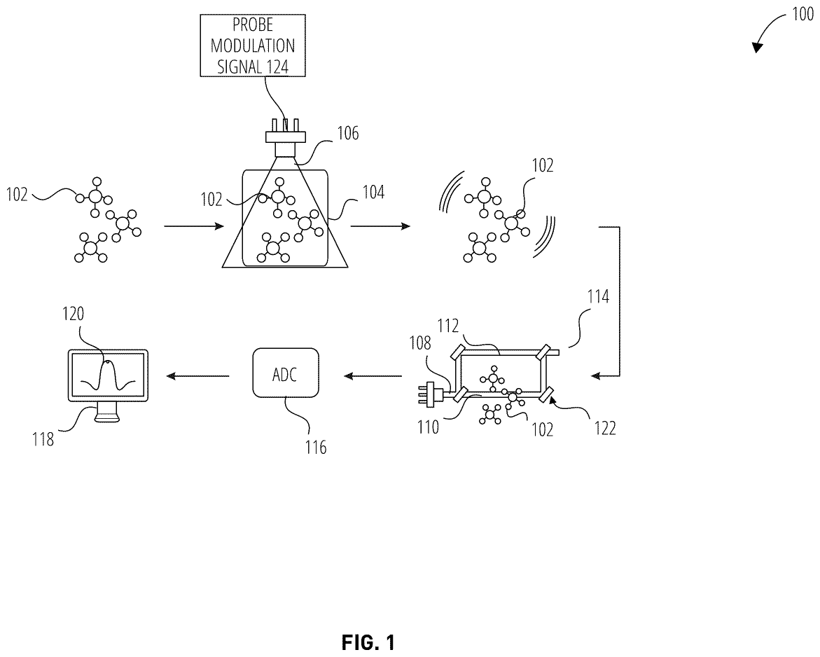

FIG. 1 illustrates conventional WMS gas sensor operation 100. During conventional WMS gas sensor operation 100, a sample under test 102 within a sample reservoir 104, such as molecules of a targeted gas, may be subject to an excitation laser 106. The power in the excitation laser 106 may heat the gas. Depending on the type of gas, light energy at certain wavelengths, in the form of photons, may be absorbed by the molecules, raising them into an excited state. In this excited state, the gas density and thus its refractive index may change.

These changes in density result in a change in the refractive index of the gas in the sample under test 102. A probe laser 108 may pass through the gas, providing a signal through which the refractive index changes may be detected and measured. In one example, the probe laser 108 may be split into two beams, one of which, a test beam 110, passes through the excited gas and the other of which, a reference beam 112, does not pass through the gas. The test beam 110 and reference beam 112 may be recombined and detected at a photodetector 114. The signal may be acquired and processed through signal acquisition 116 and signal processing 118 techniques which are well known in the art to arrive at sample concentration data 120.

Synchronous detection using a lock-in amplifier may be used to detect and measure weak signals buried in noise. This technique involves modulating the interferometric signal and then using a reference signal to extract the desired information. Fast Fourier Transform (FFT) may be used. By converting a time-domain signal into its frequency components, the periodic interference patterns may be identified and analyzed, and selective frequency noise may be thrown away. Phase unwrapping may be used to determine the continuous phase of an interferometric signal. Since the measured phase is often wrapped between −π and π, unwrapping helps in understanding the true phase changes, which are crucial for accurate gas concentration measurements.

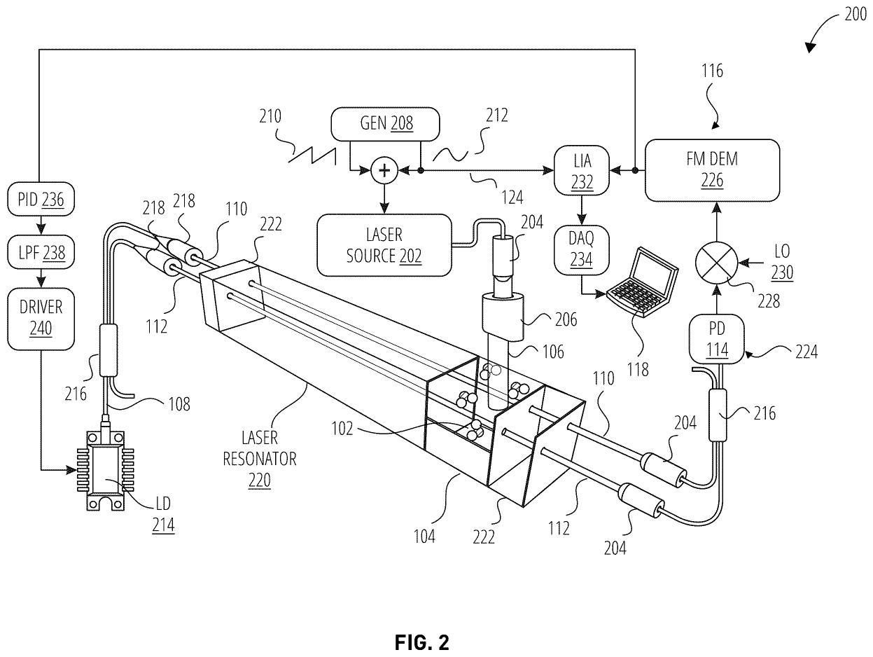

A Mach-Zehnder interferometer 122 configuration may be used to split the reference beam 112 from the test beam 110, pass the test beam 110 through the sample under test 102, and combine the resulting test beam 110 and reference beam 112 for detection at the photodetector 114. A beat node detector configuration may also be used, as is shown by the beat node detector 224 introduced in FIG. 2. Mach-Zehnder architecture relies upon phase shifting as the beams travel the two paths. Upon recombination, the phase differences between the two signals may cause the beams to combine with constructive or destructive interference. In this manner, changes to the refraction of the test beam 110 caused by changes in the sample under test 102 may be measured in the signal detected by the photodetector 114.

By applying a probe modulation signal 124, the wavelength of the excitation laser may be modulated. WMS uses this modulation to imbed discrete frequencies in the detector signal while the nominal laser wavelength is scanned across a molecule's absorption range. The modulation may shift absorption information to harmonics of the modulation frequency, which may then be extracted using lock-in filters in signal processing. This technique may provide a high degree of accuracy under challenging test conditions. However, the excitation laser 106 may typically be a solid-state laser. Solid-state lasers may have problems performing under the desired signal modulation conditions. Solid-state lasers may also consume more power than is generally feasible in the types of portable applications desired. Other types of lasers, such as semiconductor lasers, may be more compact, consume less energy, and have more easily modulated output, but may not be able to adequately excite the sample under test 102.

FIG. 2 illustrates a conventional WMS gas sensor 200 which may perform a process similar to the conventional WMS gas sensor operation 100 described above. The sample under test 102 may reside in a sample reservoir 104 that is an 8 mm³ volume and may in a portion of that region be subject to an excitation laser 106 such as may be generated by a 2 μm laser source 202 passed through a fiber-pigtailed collimator 204 and a cylindrical lens 206. A signal generator 208 (GEN) may provide a probe modulation signal 124, including a ramp signal 210 and a lock-in sine wave 212.

The probe laser 108 may be laser diode 214 (LD), which may be an 808 nm pump laser diode 214. The signal from the probe laser 108 may be split by a fiber coupler 216 to form the test beam 110 and reference beam 112. The probe laser 108 signal and reference beam 112 may be directed through two gradient refractive index (GRIN) lenses such as GRIN lenses 218 into a laser resonator 220 formed from an amplifying medium such as yttrium orthovanadate (YVO₄) with dielectric, 1,064 nm laser mirrors 222 at each end.

The reference beam 112 may pass through a volume of the sample under test 102 that is not affected by the excitation laser 106 while the test beam 110 passes through the excited portion of the sample under test 102. Both may be collected by fiber-pigtailed collimators 204, coupled through fiber coupler 216, and detected as input to a silicon photodetector 114 (PD) that in this example is part of a beat node detector 224 rather than a Mach-Zehnder interferometer 122.

Signal acquisition 116 may employ a radio frequency (RF) mixer 228 (MIX), a frequency modulation demodulator 226 (FM DEM), a local oscillator signal 230 (LO), a lock-in amplifier 232 (LIA), and a data acquisition card 234 (DAQ), which may transmit data to a computational device for signal processing 118. The output from the frequency modulation demodulator 226 may be sent to a three-term controller 236 such as a proportional-integral-derivative (PID) controller, thence to a low-pass filter 238 (LPF), and on to a driver circuit 240 for the laser diode 214, as shown. The sample concentration data 120 may be detectable from the resulting 2f WMS signal 300.

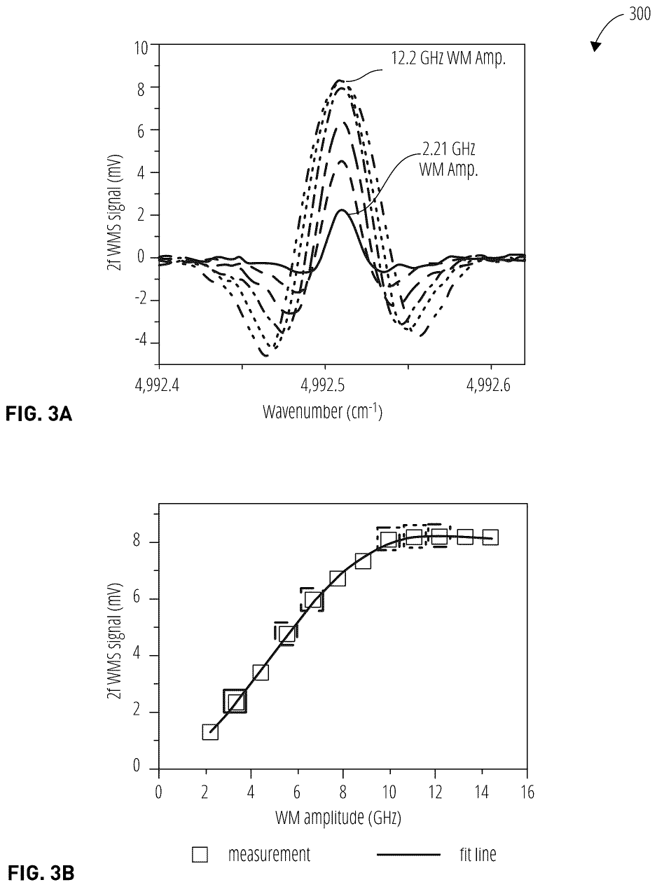

FIG. 3A illustrates a 2f WMS signal 300 registered for 1000 ppm of CO₂ plotted for increasing sinewave modulation amplitude. FIG. 3B shows the maximum amplitude of the signal registered at the transition center. Line dashing around the wavelength modulation (WM) amplitude measurements of FIG. 3B correspond to the signals shown in FIG. 3A.

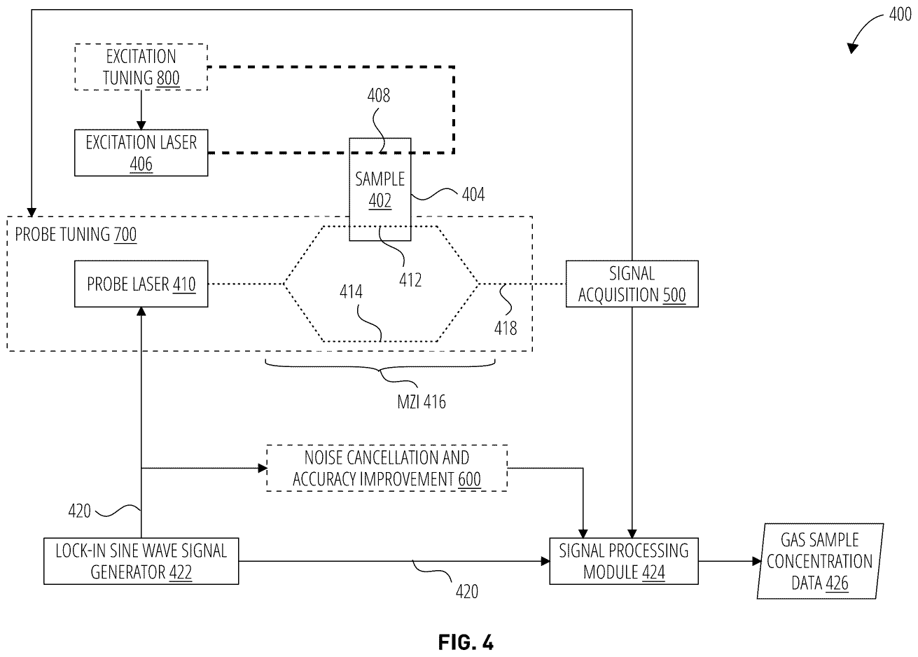

FIG. 4 illustrates the disclosed photonic gas sensor 400 in accordance with one embodiment. This photonic gas sensor 400, based on infrared laser technology, may be used to measure the concentrations of different gases. The photonic gas sensor 400 may operate in a manner similar to the conventional WMS gas sensor operation 100 described above with respect to FIG. 1.

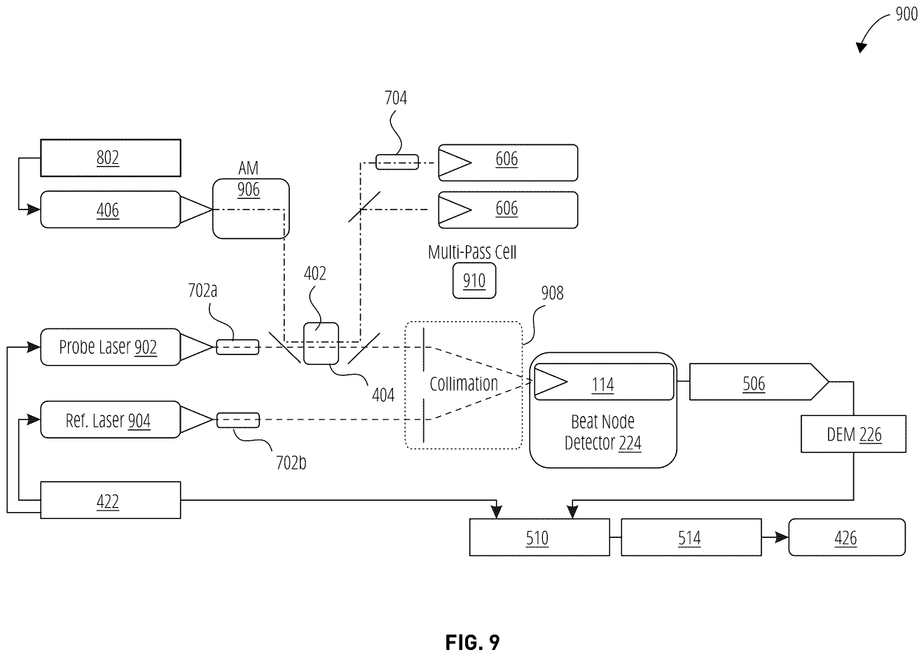

An excitation laser 406, otherwise referred to herein as a pump laser, may transmit an excitation beam 408 onto a gas sample under test 402 in a sample reservoir 404 to heat up or excite the molecules of the gas sample under test 402. A probe laser 410 may be split to form a test beam 412 and a reference beam 414. The test beam 412 may be passed through the gas sample under test 402 and recombined with the reference beam 414 to form a recombined signal 418 using an interferometer such as a Mach-Zehnder interferometer 416. In one embodiment, the sample reservoir 404 may be a multi-pass cell, such as the resonator shown in FIG. 2. In one embodiment a multimode interferometer, Jamin interferometer, or other such structure may be used. In one embodiment, beat node architecture may be used in place of Mach-Zehnder architecture, similar to the configuration shown for the conventional WMS gas sensor 200. Such a configuration of the disclosed sensor is illustrated in and described in greater detail with respect to FIG. 9. The recombined signal 418 may be detected at signal acquisition 500, for example by at a photodetector 502. The output of the photodetector 502 may be processed by signal processing module 424 to arrive at gas sample concentration data 426.

However, in contrast to the conventional approaches shown for conventional WMS gas sensor operation 100 and conventional WMS gas sensor 200 and described above, the photonic gas sensor 400 of the present disclosure overcomes many of the limitations of those conventional approaches, such as issues with sensitivity, reliability, cost, and size. The photonic gas sensor 400 achieves these improvements by omitting the wave modulation of the excitation laser and instead applying a probe modulation signal 420 to the probe laser 410. Because the probe laser 410 does not need the power capabilities to heat or excite the gas sample under test 402, a semiconductor laser may be used. These lasers are small, inexpensive, and offer easy signal modulation capabilities.

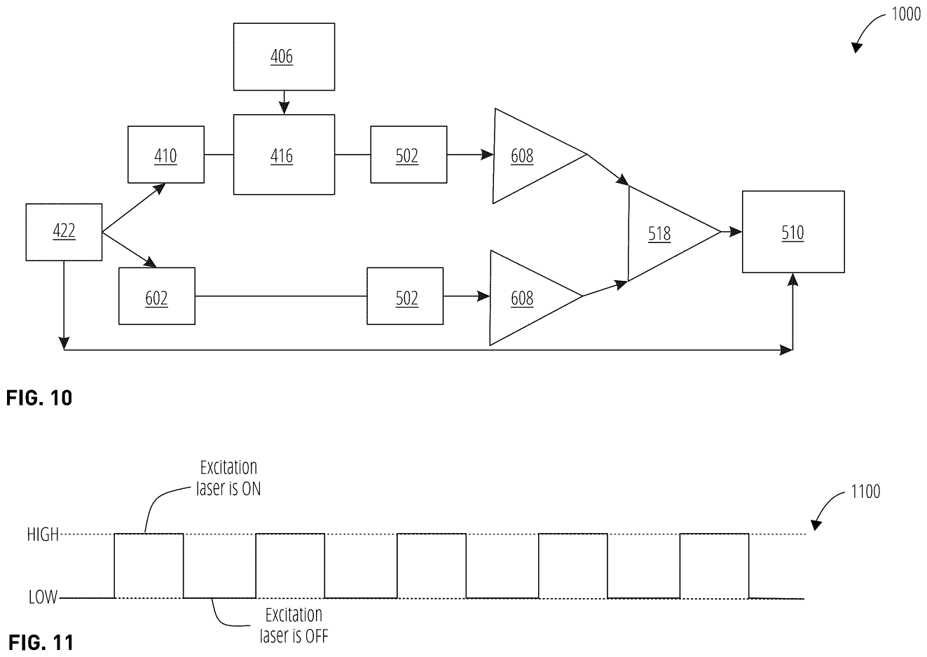

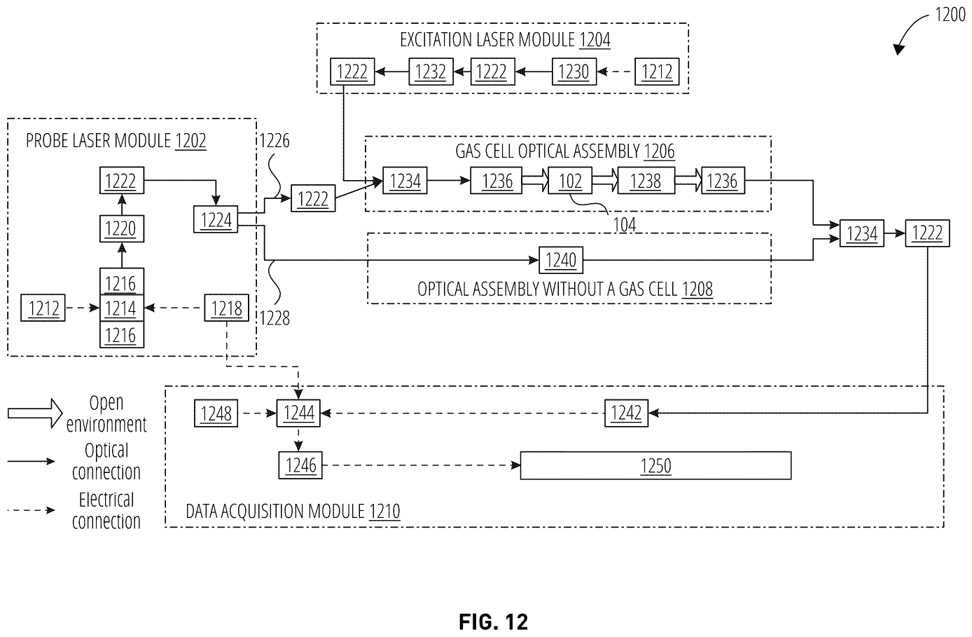

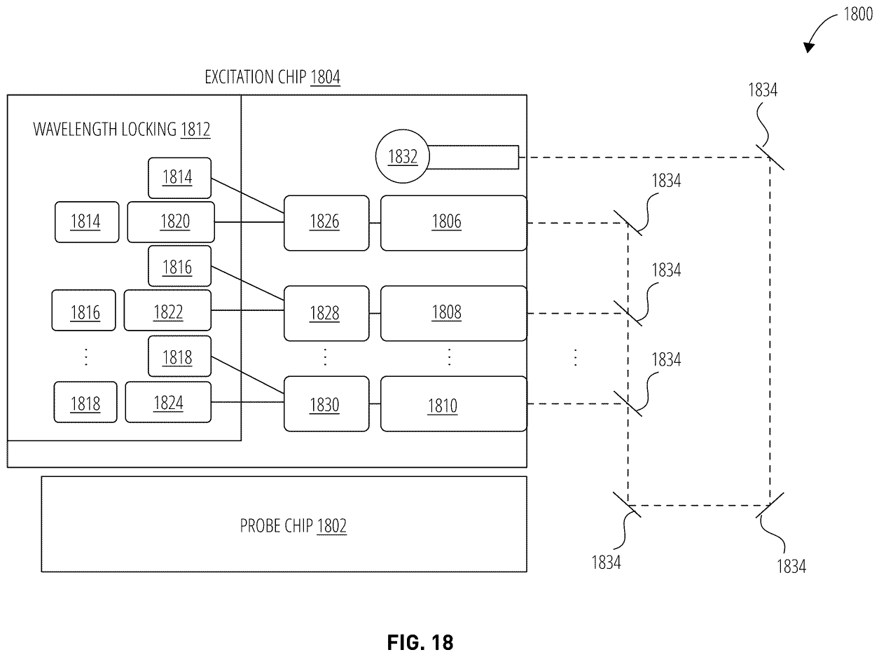

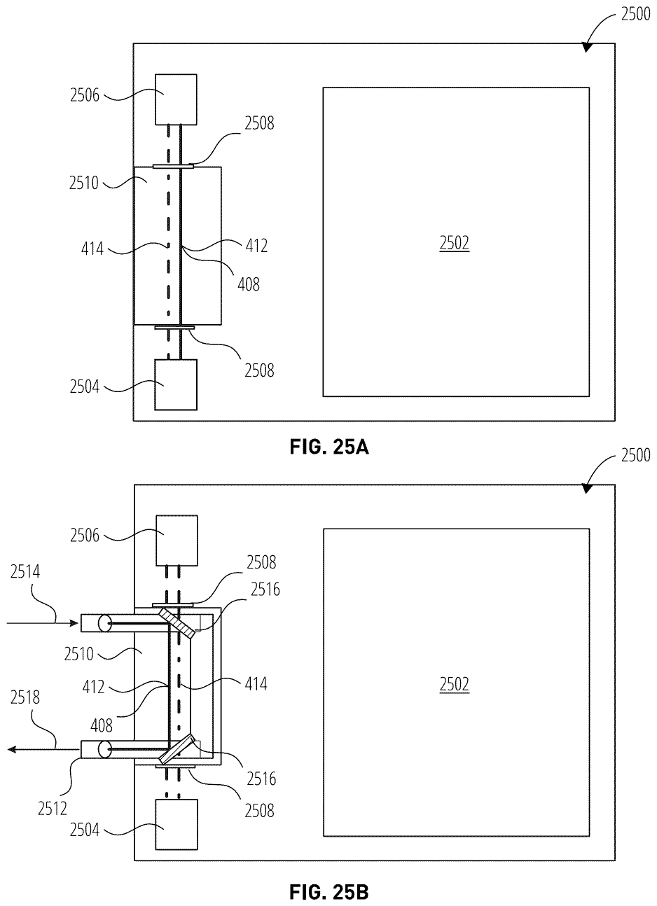

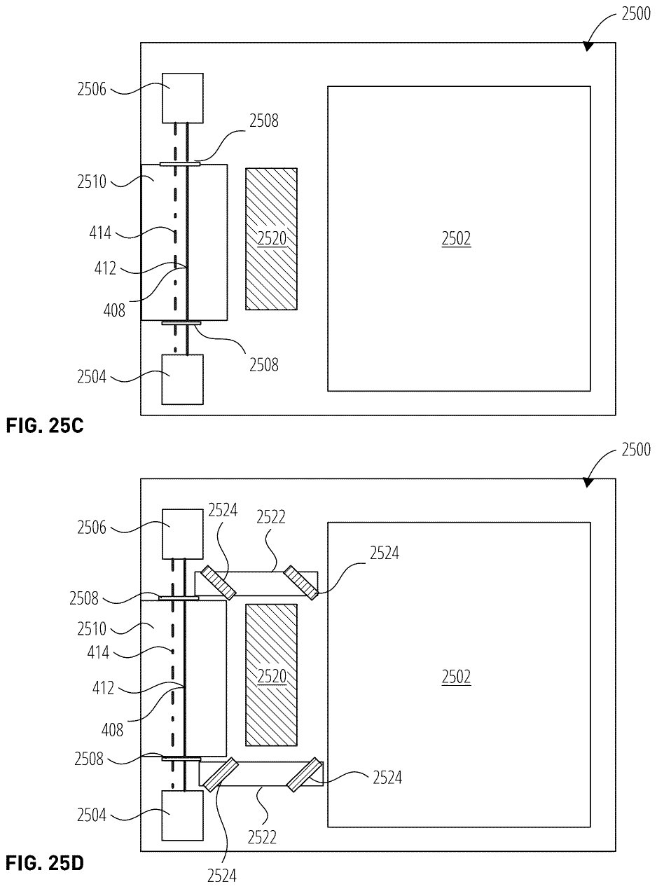

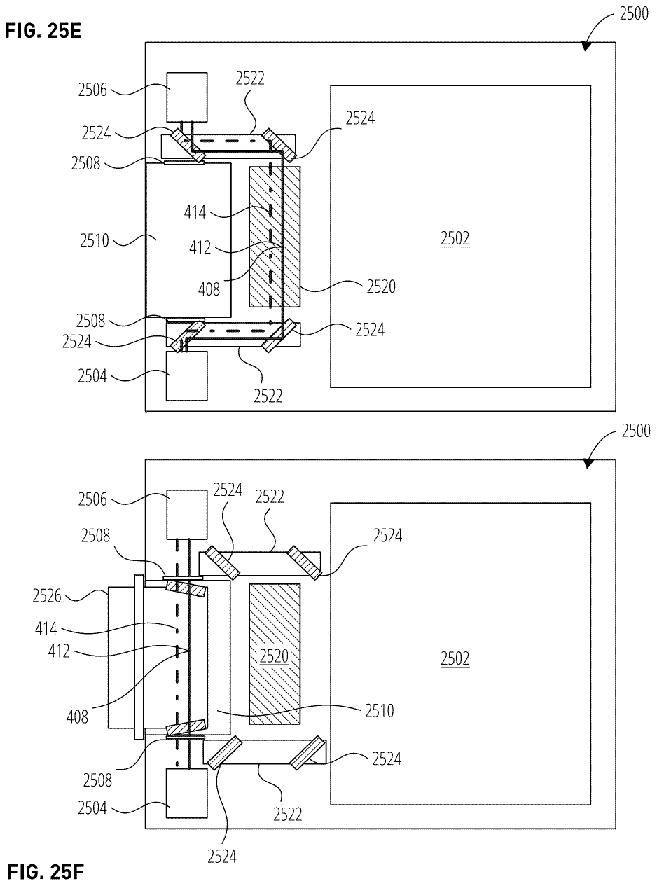

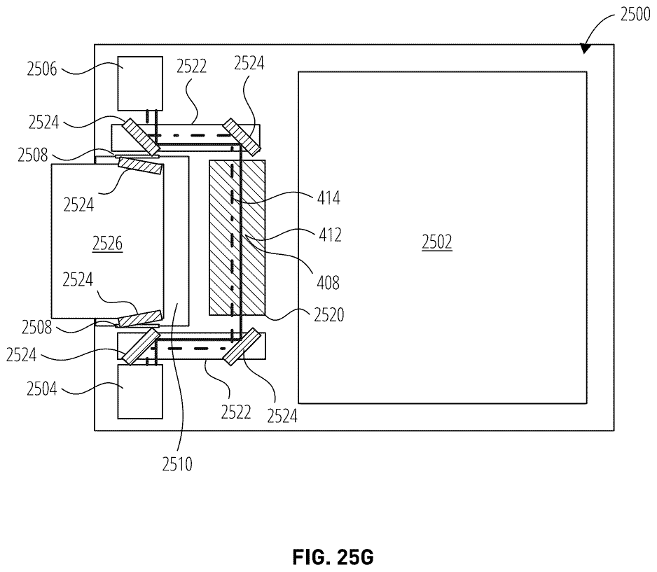

Similar signal acquisition 500 and signal processing module 424 techniques well known in the art may be used to develop gas sample concentration data 426 from the output of the photonic gas sensor 400 as detected by the photodetector 502 and from the attributes of the probe modulation signal 420. Noise cancellation and accuracy improvements 600, probe signal tuning 700, and excitation signal tuning 800 may further refine the already high accuracy of the results that may be achieved by the disclosed solution. These are described in greater detail below. Additional embodiments for photonic gas sensors are illustrated in FIG. 9, FIG. 10, FIG. 12-FIG. 16, FIG. 18-FIG. 20, and FIG. 25A-FIG. 25G.

Synchronous modulation in the disclosed solution thus be performed via the probe laser 410 and, in one embodiment, the offset laser with inverted lock-in signal, described in more detail with respect to FIG. 6. In one embodiment, synchronous modulation may be employed as follows:
1310 nm probe laser:
   MZI path length difference small but for instance <100 μm
   Wavelength tuning for max slope of interference signal
   Modulation small defined by MZI path length difference
   Wavelength change compensated by phase change in photonic integrated chip
1530 nm excitation laser:
   Preferably constant power with small modulation depth <10%
   Conversion of imaginary refractive index (absorption) to real refractive index (optical path, phase change)

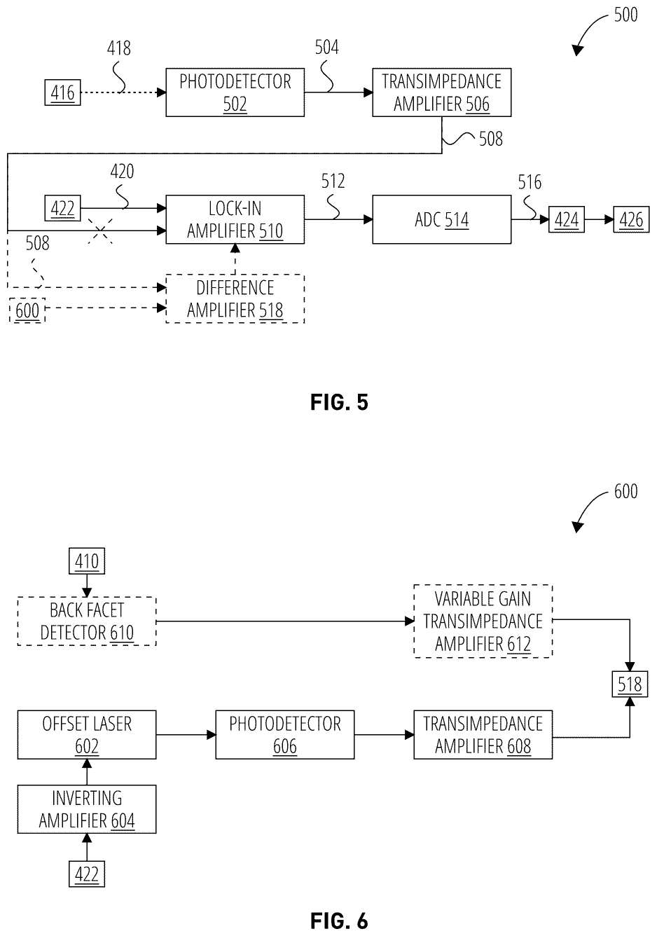

FIG. 5 illustrates signal acquisition 500 in accordance with one embodiment. The signal acquisition 500 may be performed by a photodetector 502, a transimpedance amplifier 506, a lock-in amplifier 510, an ADC 514, and a difference amplifier 518.

A photodetector 502 such as a photodiode may detect the recombined signal 418 output of the Mach-Zehnder interferometer 416. The detected recombined signal 504 generated by the photodetector 502 may be passed through a transimpedance amplifier 506. The transimpedance amplifier 506 may convert the weak current detected recombined signal 504 from the photodetector 502, in a destructive state of interference, into a voltage signal 508 with sufficient gain to provide an improved signal to noise ratio (SNR) and better overall signal quality. The voltage signal 508 may then be suitable for the signal processing module 424 steps which may be performed to interpret data carried in the voltage signal 508 regarding the sample under test.

The voltage signal 508 output of the transimpedance amplifier 506 (a sensing signal) may be sent to the lock-in amplifier 510 for comparison with the probe modulation signal 420 from the lock-in sine wave signal generator 422 (a reference signal), which both modulate at the same frequency. The lock-in amplifier 510 may then synchronously extract the effect of excitation on the gas sample under test 402, free of other frequency noise, in the form of the lock-in amplifier output signal 512.

The lock-in amplifier output signal 512 may be converted to a digital output signal 516 by the ADC 514. Examination of this digital signal using signal processing module 424 techniques may yield gas sample concentration data 426 pertaining to the gas sample under test 402, based on its behavior when exposed to the excitation beam 408 from the excitation laser 406 and the test beam 412 from the probe laser 410.

In another embodiment, additional signals may be used to eliminate noise. In such embodiments, the output voltage signal 508 from the transimpedance amplifier 506 may be sent to a difference amplifier 518 instead of directly to the lock-in amplifier 510. The amplified difference between the signals sent to the difference amplifier 518 may represent a cleaner, lower-voltage signal, as is explained in greater detail below.

FIG. 6 illustrates noise cancellation and accuracy improvement 600 in accordance with one embodiment. The output of the Mach-Zehnder interferometer 416 may have a direct current (DC) component that carries no information about the gas sample under test 402. Reducing this portion of the signal may allow a lower-cost ADC 514 to be used or may allow higher resolution to be achieved with the same ADC 514. An offset laser 602 may be used as described below to remove the DC component from the signal sent for signal processing module 424.

The probe modulation signal 420 from the lock-in sine wave signal generator 422 may be inverted by the inverting amplifier 604 and may be transmitted by an offset laser 602. A photodetector 606 and transimpedance amplifier 608 similar to the photodetector 502 and transimpedance amplifier 506 used for signal acquisition 500 may be used to detect the signal from the offset laser 602 and convert it from current to voltage. The signals from both the probe laser 410 and the offset laser 602 may be processed through the difference amplifier 518. In this manner, the DC component of the signals may be eliminated.

In one embodiment, a signal from a back facet detector 610 configured as part of the probe laser 410 may be amplified using an adjustable variable gain transimpedance amplifier 612 in order to compensate for the offset. Commercial lasers may have an inbuilt monitoring photodiode on the backside of the laser cavity that acts as the back facet detector 610. The signal detected by the back facet detector 610 may have the same correlated noise characteristics as the light that goes through the interferometer.

The monitor photodiode or back facet detector 610 signal may be used, after amplification, to subtract unwanted signal artifacts electrically from the final interfered output signal using the difference amplifier 518. This may allow variations of noise from laser power drift and instability to be canceled out in addition to reducing the voltage level of the signal.

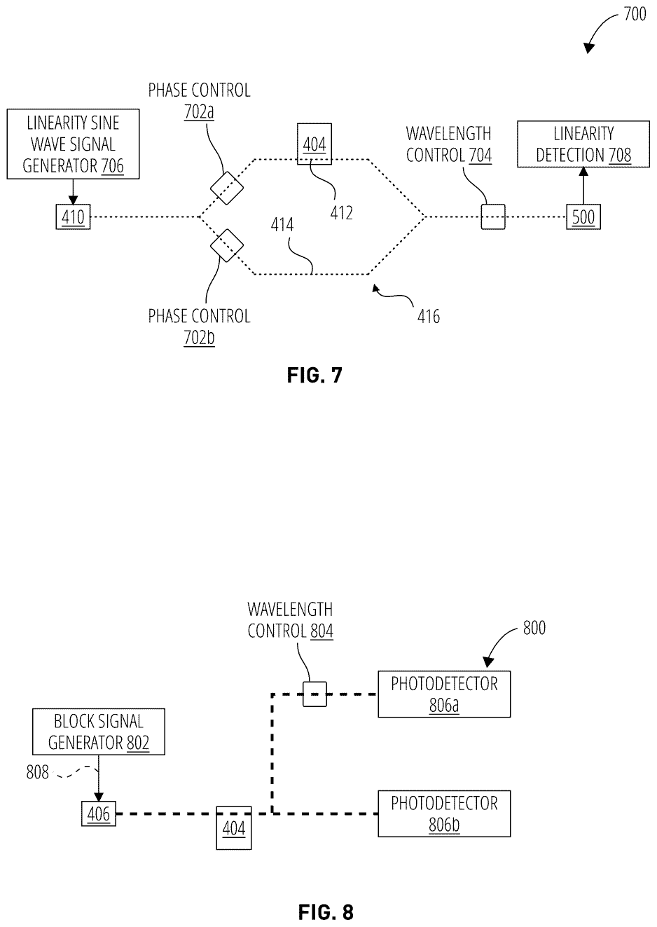

FIG. 7 illustrates probe signal tuning 700 in accordance with one embodiment. Probe signal tuning 700 may include phase control 702a and phase control 702b, wavelength control 704, and linearity monitoring using a linearity sine wave signal generator 706 and linearity detection 708.

Phase control 702a and phase control 702b of the test beam 412 and reference beam 414 respectively in the Mach-Zehnder interferometer 416 may allow independent phase adjustments that may help reduce noise introduced by asymmetry in the Mach-Zehnder architecture (MZA), or may allow asymmetrical MZA to be used in applications where it may be beneficial without having to filter undesirable effects out of the data in post-processing.

MZIs operate on waveform properties whereby phase shifts in a waveform traveling down one leg lead to constructive or destructive interference when combined with a similar waveform traveling down the other leg. In one embodiment, phase control may allow a zero-point to be established at the photodetector 502. That is, the phase shifts may be tuned such that the waveform passing through the gas sample under test 402, i.e., the test beam 412, may cancel the reference beam 414. If, for example, a zero point is achieved when the excitation laser 406 is off and the gas sample under test 402 is unexcited, then excitation of the gas sample under test 402 by the excitation laser 406 would result in a detectable waveform at the photodetector 502. In one embodiment, the phase control 702a and phase control 702b may be used to adjust the Mach-Zehnder interferometer 416 during a calibration step to reduce background noise.

Probe signal tuning 700 may also incorporate wavelength control 704. Wavelength control 704 at the output of the Mach-Zehnder interferometer 416 may be one port of a 1*2 splitter used to optically monitor the wavelength of the Mach-Zehnder interferometer 416 output signal. This may allow active compensation for unwanted phase differences in the system by changing the wavelength of the probe laser 410. In this manner, the quadrature point of the Mach-Zehnder interferometer 416 may be maintained at a fixed level for maximum sensitivity and repeatability. In one embodiment, wavelength control 704 may be an electrical signal after the photodetector 502 and before the transimpedance amplifier 608.

A sine wave may be added to the signal carried by the probe laser 410 by the linearity sine wave signal generator 706. This signal component may be detectable by the linearity detection 708. A small signal from the linearity sine wave signal generator 706 may be continuously transmitted by the probe laser 410 through the Mach-Zehnder interferometer 416. This signal may form harmonics when the Mach-Zehnder interferometer 416 is tuned away from the zero point. These harmonics may be detected by linearity detection 708 when the excitation laser 406 is off. The Mach-Zehnder interferometer 416 may be tuned using phase control 702a and phase control 702b as described above to compensate for any drift in the quadrature point of the Mach-Zehnder interferometer 416.

FIG. 8 illustrates excitation signal tuning 800 in accordance with one embodiment. Excitation signal tuning 800 may involve monitoring the excitation laser 406 and adjusting how it is driven by a block signal generator 802 based on wavelength control 804 and detections at photodetector 806a and photodetector 806b.

The block signal generator 802 may provide signaling control for the excitation laser 406 in the form of a block signal that operates the excitation laser 406 at a desired excitation frequency during blocks of time. When the block signal is high, the excitation laser 406 may affect the gas sample under test 402, which will absorb energy and heat up at a wavelength characteristic of the molecular structure of the sample. For example, as described previously, CO₂ may heat up when a 2 μm signal is applied.

While the block signal is high and the excitation laser 406 is on, wavelength control may be applied in the excitation laser 406 to ensure the correct absorption wavelength for the gas sample under test 402 is maintained. The measurement range may be adjusted, and measurements may be made based on signals detected by the photodetectors. While the block signal is low and the excitation laser 406 is off, linearity checks may be performed to ensure the quadrature point of the Mach-Zehnder interferometer 416 is set as desired using phase control 702a and phase control 702b, by changing the wavelength of the probe laser 410, etc. An exemplary block signal 1100 is illustrated in FIG. 11.

In one embodiment, the block signal generator 802 may provide an excitation modulation signal 808 to the excitation laser 406. This may support operating the photonic gas sensor 400 with collimated beams having a small diameter. This may also minimize the effects of optical cavities when various optical elements are positioned at an angle relative to the beams.

After passing through the gas sample under test 402, the excitation laser 406 may be split into two beams. One beam may have wavelength control 804 applied to ensure the wavelength does not drift from the absorption peak of the gas sample under test 402. This may be detected at photodetector 806a. The other beam may be detected at photodetector 806b to safely sink the high-power light.

FIG. 9 illustrates a photonic gas sensor 900 in accordance with one embodiment. The photonic gas sensor 900 may utilize many components similar to those used in the photonic gas sensor 400 introduced in FIG. 4. The photonic gas sensor 900, however, may not use the same WMS techniques, and may instead rely upon a beat node detector 224 similar to that described with respect to FIG. 2, in addition to other elements that represent a technological improvement over the conventional WMS gas sensor 200.

In contrast to the photonic gas sensor 400, the photonic gas sensor 900 may utilize a separate probe laser 902 and reference laser 904. The excitation laser 406 may undergo amplitude modulation 906. The beams of the probe laser 902 and reference laser 904 may undergo collimation 908 after the probe laser 902 has passed through the gas sample under test 402 and before the collimated laser signal is received by the photodetector 114 of a beat node detector 224. The photonic gas sensor 900 may in addition include a multi-pass cell 910.

The collimation 908 may produce collimated beams which may be capable of providing a longer distance of overlap between the probe laser 902 and excitation laser 406 beams. This may result in more efficient heating of the gas sample under test 402 as well as more efficient probing of the sample. The multi-pass cell 910 may extend the effective length of the probe laser 902 and excitation laser 406 beams within the gas sample under test 402. Gas absorption is related to the length of the gas light interaction. By reflecting the beams back and forth several times while shifting every cycle a small distance and ending the reflections at a point such that the last reflection encounters the collimation 908 and beat node detector 224 in a defined manner, a longer beam length may be achieved within the gas sample under test 402 without enlarging the sensor device.

While the signal from the block signal generator 802 is low, the photonic gas sensor 900 may perform an optical power check, prove wavelength tuning, and reference wavelength tuning. While the block signal is high, the photonic gas sensor 900 may perform wavelength control, measurement, and wavelength check operations. An exemplary block signal 1100 is illustrated in FIG. 11.

FIG. 10 illustrates a photonic gas sensor 1000 in accordance with one embodiment. The photonic gas sensor 1000 may comprise elements similar to those already introduced with respect to the photonic gas sensor 400 illustrated in FIG. 4-FIG. 6. The photonic gas sensor 1000 may involve offset compensation via splitting of probe laser 410 signal and a difference amplifier 518, as indicated in FIG. 5. The probe laser 410 may be split into a test beam 412 and a reference beam 112 as previously described or may be provided by two separate laser diodes operating at the same frequency. The test beam 412 and reference beam 414 may in one embodiment be from a 1,310 nm laser. In contrast, the excitation laser 406 may be a 1,530 nm laser.

While the block signal driving the excitation laser is low (the excitation laser 406 if off, differential amplifier nulling may be performed. An exemplary block signal 1100 is illustrated in FIG. 11.

FIG. 11 illustrates a block signal 1100, which may be used to drive an excitation laser in embodiments of the photonic gas sensor such as those described above. When the signal is high, the excitation laser may be turned on and testing to measure a concentration of a gas sample may be performed. When the block signal 1100 is low, the excitation laser may be OFF, and various turning and calibration activities may be performed.

FIG. 12 illustrates a photonic gas sensor 1200 in accordance with one embodiment. The photonic gas sensor 1200 may include a probe laser module 1202, an excitation laser module 1204, a gas cell optical assembly 1206, an optical assembly without a gas cell 1208, and a data acquisition module 1210.

The probe laser module 1202 may include a laser and thermoelectric cooler controller 1212, a modulator 1214, at least one modulator connection board 1216, a function generator 1218, a 1,310 nm laser 1220, an optical fiber connector 1222, and a 90/10 splitter 1224. The laser and thermoelectric cooler controller 1212 may be electrically coupled to a modulator 1214, the modulator 1214 having two modulator connection boards 1216. A function generator 1218 may also be electrically connected to the modulator 1214. The modulator 1214 may, through one modulator connection board 1216, transmit a 1,310 nm laser 1220 through a an optical fiber connector 1222 to a 90/10 splitter 1224. The 90% part 1226 of the 90/10 splitter 1224 may transmit the laser through an optical fiber connector 1222 to the gas cell optical assembly 1206. The 10% part 1228 from the 90/10 splitter 1224 may be transmitted to the optical assembly without a gas cell 1208.

The excitation laser module 1204 may comprise a laser and thermoelectric cooler controller 1212, a 1,530 nm laser 1230, optical fiber connectors 1222, and an erbium doped fiber amplifier 1232. The laser and thermoelectric cooler controller 1212 may control a 1,530 nm laser 1230, which may travel through an optical fiber connector 1222 to the erbium doped fiber amplifier 1232. The laser may then travel through an optical fiber connector 1222 to the gas cell optical assembly 1206.

The gas cell optical assembly 1206 may include an optical combiner 1234, optical collimators 1236, a sample reservoir 104 containing a sample under test 102, and a 45° dichroic mirror 128. The 90% part 1226 of the 1,310 nm laser 1220 from the probe laser module 1202 and the 1,530 nm laser 1230 from the excitation laser module 1204 may be directed to the optical combiner 1234 of the gas cell optical assembly 1206. The optical combiner 1234 may be optically coupled to an optical collimator 1236. Collimated beams from the optical collimator 1236 may pass through an open environment, such as air, vacuum, another gaseous medium, etc., through the sample reservoir 104 containing the sample under test 102. The beams may again pass through an open environment to the 45° dichroic mirror 1238 and thence to an optical collimator 1236.

The output of the optical collimator 1236 (which is the output of the gas cell optical assembly 1206, may be collected at an optical combiner 1234. The optical assembly without a gas cell 1208 may include a polarization controller 1240 optically coupled to receive the 10% part 1228 of the 1,310 nm laser 1220 from the 90/10 splitter 1224. The output of the polarization controller 1240 may then be optically coupled to the optical combiner 1234. The output of the optical combiner 1234 may travel through an optical fiber connector 1222 to the data acquisition module 1210.

The data acquisition module 1210 may include a photodiode 1242, a lock-in amplifier 1244, an analog to digital converter 1246, a direct current power supply 1248, and a computing system 1250. The photodiode 1242 may receive the output of the optical combiner 1234 through the optical fiber connector 1222 as shown. The photodiode 1242 may be electrically coupled to provide its output to the lock-in amplifier 1244. The lock-in amplifier 1244 may also receive a signal through electrical coupling to the function generator 1218 of the probe laser module 1202. The lock-in amplifier 1244 may be powered by the direct current power supply 1248. The output of the lock-in amplifier 1244 may be converted to a digital signal by the analog to digital converter 1246. This digital output may be provided to the computing system 1250 for data processing and storage. In one embodiment, the computing system 1250 may be a miniature computer such as a Raspberry Pi. In one embodiment, the analog to digital converter 1246 may be provided as part of an electronics board such as an Arduino.

The probe laser module 1202 of the photonic gas sensor 1200 may in one embodiment use a probe laser signal and not a signal with wavelength for absorption. The excitation laser may in one embodiment be a fine-tunable laser with an amplifier, such as an erbium doped fiber amplifier 1232 (EDFA) or a semiconductor amplifier. In one embodiment, a broadband tunable laser may be used to switch between different absorption wavelengths for different gases. The excitation laser module 1204 may be provide an excitation laser at power levels at or above 25 mW.

The probe laser may be a distributed-feedback laser. In one embodiment, the probe laser may be a squeezed laser with entangled photons, which may provide a 10 dB improvement in sub-nanosecond lasers.

Reference may be made to the following publications:
PINTO, Davide, et al., “Parts-per-billion detection of carbon monoxide: A comparison between quartz-enhanced photoacoustic and photothermal spectroscopy,” Photoacoustics 22 (2021).
XU, Cong, et al., “Alignment tolerant analysis of a compact 4×25 Gbps TOSA with a thin-film filter multiplexer,” Optics Communications 507 (2022).

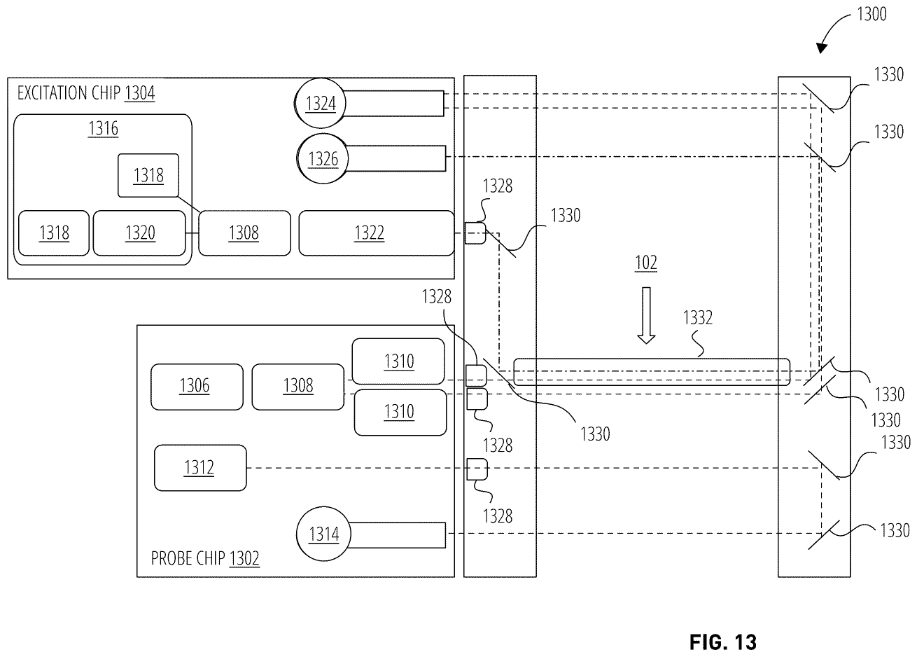

FIG. 13 illustrates a chip-based photonic gas sensor 1300 in accordance with one embodiment. The chip-based photonic gas sensor 1300 may be an ammonia sensor or other gas sensor using full photonic chip architecture, including a probe chip 1302 and an excitation chip 1304. The probe chip 1302 may include a probe laser 1306, a multimode interferometer 1308, phase control 1310, an offset laser 1312, and an offset photodetector 1314 in one embodiment. The excitation chip 1304 may include a wavelength locker 1316 including wavelength detectors 1318 and an asymmetric MZI (aMZI 1320), a multimode interferometer 1308, an excitation laser 1322, a probe photodetector 1324, and an excitation photodetector 1326.

The beam from the probe laser 1306 may pass through a multimode interferometer 1308 where it is split into two beams. The two beams may each pass through phase control 1310, and from thence through a configuration of lenses 1328 to encounter a sample under test 102 through a multi-pass cell 1332. The output from the multi-pass cell 1332 may be redirected by a configuration of reflectors 1330 to the probe photodetector 1324. Similarly, the excitation laser 1322 may pass through a lens 1328 and be reflected by reflectors 1330 to pass through the multi-pass cell 1332 and excite the sample under test 102. The beam from the excitation laser 1322 may then be reflected to the excitation photodetector 1326 as shown. The beam from the offset laser 1312 may be transmitted through a configuration of lenses 1328 and reflectors 1330 to be collected at the offset photodetector 1314.

The excitation chip 1304 may be configured with a wavelength locker 1316 for the excitation laser 1322. The excitation laser 1322 may send a signal to a multimode interferometer 1308, which may spit the signal, sending it to a wavelength detector 1318 so as to measure the reference signal, as well as to an aMZI 1820 the output of which is sent to another wavelength detector 1318.

In one embodiment, the photonic integrated circuit may include one probe laser, one monitor diode, phase control sections for each MZI waveguide, an aZMI for wavelength control (locker), a photodiode for measuring the aZMI output, a photodiode for measuring a reference from the MZI wavelength locker, and a photonic integrated circuit (PIC) controller control circuit to control the MZI waveguide phase to compensate for phase change for wavelength shift under modulation.

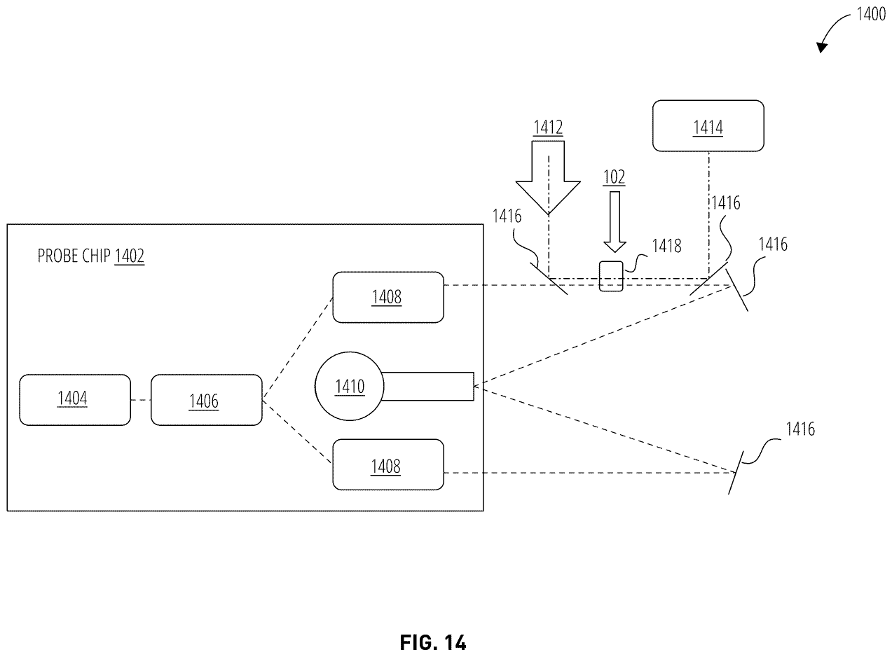

FIG. 14 illustrates aspects of a probe chip 1402 of a chip-based photonic gas sensor 1400 in accordance with one embodiment. In one embodiment, the chip-based photonic gas sensor 1400 may be configured as an ammonia sensor and may use an MZI photonic chip architecture.

The chip-based photonic gas sensor 1400 may examine a sample under test 102 using a probe chip 1402 which includes a probe laser 1404, a multimode interferometer 1406, phase control 1408, and an output photodetector 1410. The multimode interferometer 1406 may split the probe laser 1404 into two beams, one of which may pass through the sample under test 102 in a multi-pass cell 1418. Reflectors 1416 may direct the beam from the multi-pass cell 1418 and the other beam back to the output photodetector 1410 for detection and analysis. An excitation laser 1412 may be directed by reflectors 1416 through a multi-pass cell 1418 to an excitation photodetector 1414, as shown.

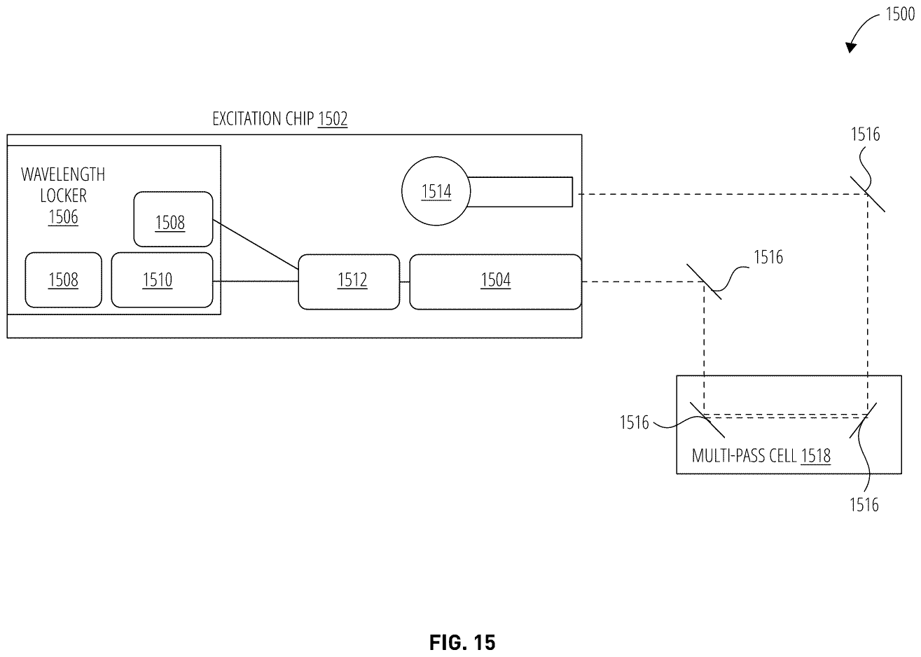

FIG. 15 illustrates aspects of an excitation chip 1502 of a chip-based photonic gas sensor 1500 in accordance with one embodiment. In one embodiment, the chip-based photonic gas sensor 1500 may be configured as an ammonia sensor using direct absorption photonic chip architecture.

The chip-based photonic gas sensor 1500 may comprise an excitation chip 1502 providing an excitation laser 1504. Wavelength locker 1506 on the excitation chip 1502 may be used as input for a multimode interferometer 1512 for the excitation laser 1504. The wavelength locker 1506 may use wavelength detectors 1508 and an aMZI 1510. reflectors 1516 may direct the excitation laser 1504 into a multi-pass cell 1518 and back to an excitation photodetector 1514 on the excitation chip 1502 for detection and analysis.

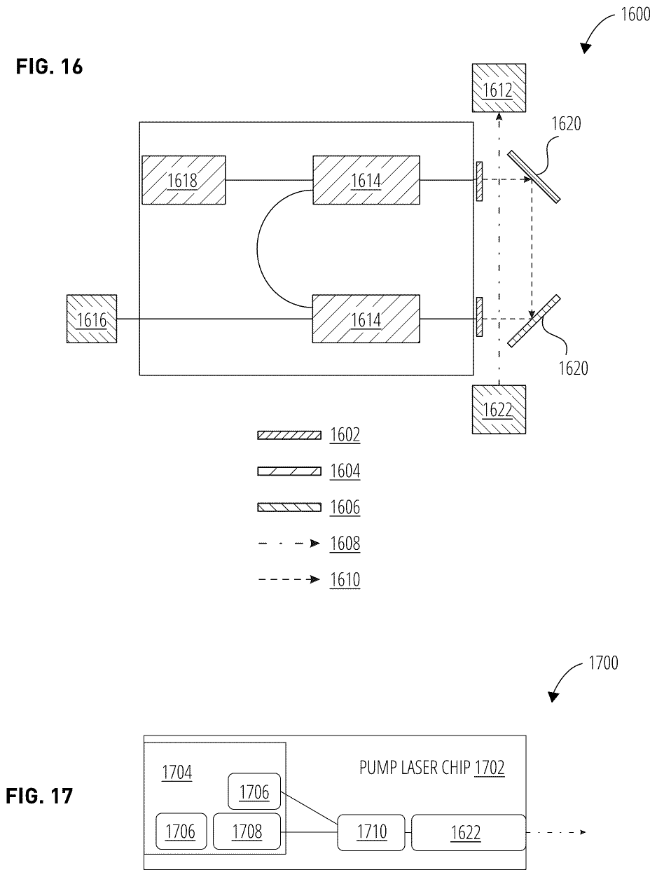

FIG. 16 illustrates a chip-based photonic gas sensor 1600 in accordance with one embodiment. The chip-based photonic gas sensor 1600 may be an ammonia sensor using a ring resonator with an air cavity and photonic chip architecture. The chip-based photonic gas sensor 1600 may include micro-lenses 1602, on-chip photonic integrated circuit blocks 1604, and discrete optics blocks 1606 to produce, detect, and analyze a sample absorption beam 1608 and a ring resonator path beam 1610. The on-chip photonic integrated circuit block 1604 may include multimode interferometers 1614 and a semiconductor optical amplifier 1618. The discrete optics blocks 1606 may include a photodetector 1612, an optical spectrum analyzer 1616, a pump laser 1622, and reflectors 1620. The pump laser 1622 may in one embodiment be a chip-based pump laser 1700 such as is illustrated in FIG. 17.

FIG. 17 illustrates a chip-based pump laser 1700 in accordance with one embodiment. The chip-based pump laser 1700 may comprise a pump laser chip 1702 configured to provide the pump laser 1622 introduced in FIG. 16. The pump laser chip 1702 may include wavelength locking 1704 including wavelength detectors 1706 and an aMZI 1708. The wavelength locking 1704 may provide outputs to a multimode interferometer 1710 to provide the pump laser 1622.

FIG. 18 illustrates a chip-based photonic gas sensor 1800 in accordance with one embodiment. The chip-based photonic gas sensor 1800 may in one embodiment be a multi-gas sensing sensor using excitation lasers sequentially switched on. The chip-based photonic gas sensor 1800 may include a probe chip 1802 and an excitation chip 1804. The probe chip 1802 may in one embodiment be the probe chip 1302 or in another embodiment the probe chip 1402 previously introduced.

The excitation chip 1804 of the chip-based photonic gas sensor 1800 may include an excitation laser 1806, an excitation laser 1808, an excitation laser 1810, and potentially additional excitation lasers configured to provide lasers at the wavelengths needed to configure the excitation chip 1804 to test the desired gas samples. The excitation chip 1804 may be configured with wavelength locking 1812 appropriate to the excitation lasers, such as the wavelength detectors 1814 and aMZI 1820 providing output to the multimode interferometer 1826 to generate excitation laser 1806, the wavelength detectors 1816, aMZI 1822, and multimode interferometer 1828 shown for excitation laser 1808, and the wavelength detectors 1818, aMZI 1824, and multimode interferometer 1830 shown for excitation laser 1810.

A number of reflectors 1834 may be configured to reflect the excitation laser beams so produced through a sample under test, and from thence to an excitation photodetector 1832 of the excitation chip 1804 for detection and analysis.

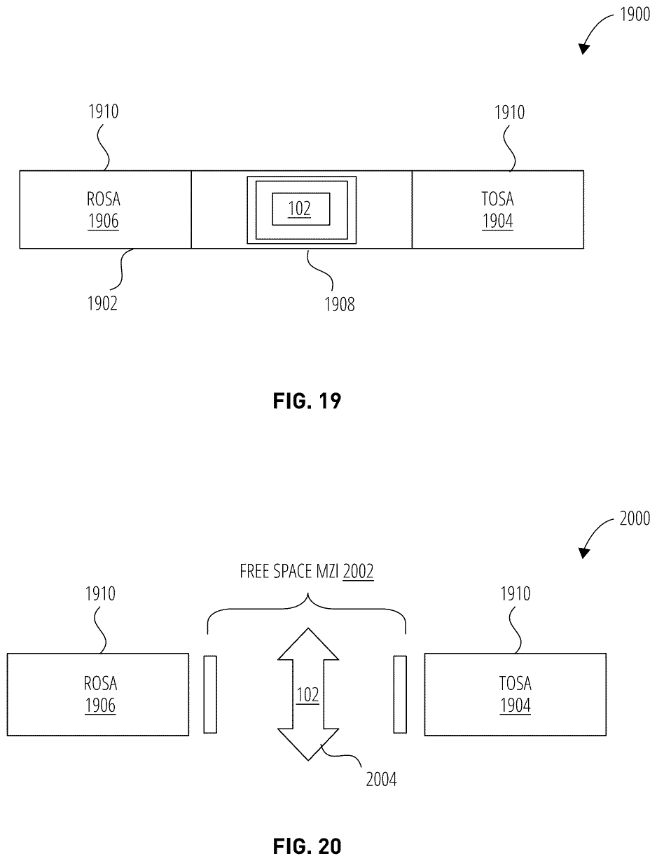

FIG. 19 illustrates a photonic gas sensor for harsh environments 1900 in accordance with one embodiment. The photonic gas sensor for harsh environments 1900 may include a frame 1902 with a transmitter optic subassembly (TOSA 1904) and a receiver optics subassembly (ROSA 1906) mounted thereon, and a transparent pipe or pipe with a window 1908 for the sample under test 102. The TOSA 1904 and ROSA 1906 may be protected by hermetic sealing 1910.

The gas flow for the sample under test 102 may flow through the transparent pipe or pipe with a window 1908 at high pressure, high concentration, high temperature, or some other condition contributing to or caused by the harsh environment. The transparent pipe or pipe with a window 1908 may be resistant to such conditions.

FIG. 20 illustrates a photonic gas sensor for harsh environments 2000 in accordance with one embodiment. The photonic gas sensor for harsh environments 2000 may include a TOSA 1904 and ROSA 1906 aligned with a free space MZI 2002. The sample under test 102 may be gas flow 2004 through the free space MZI 2002.

The gas flow 2004 may be at high pressure, high concentration, high temperature, or some other condition contributing to or caused by the harsh environment. In one embodiment, the gas flow 2004 may be through a tube resistant to such conditions.

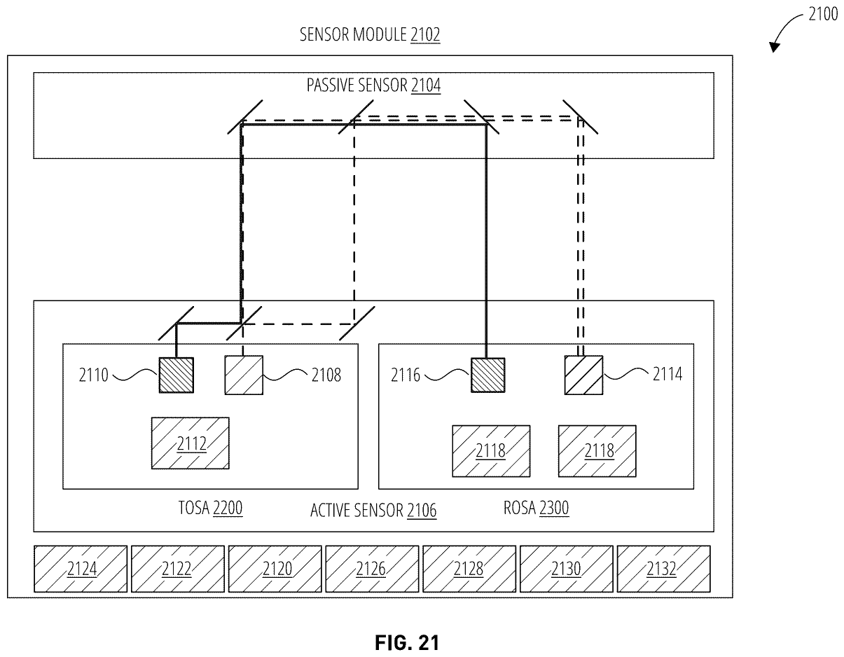

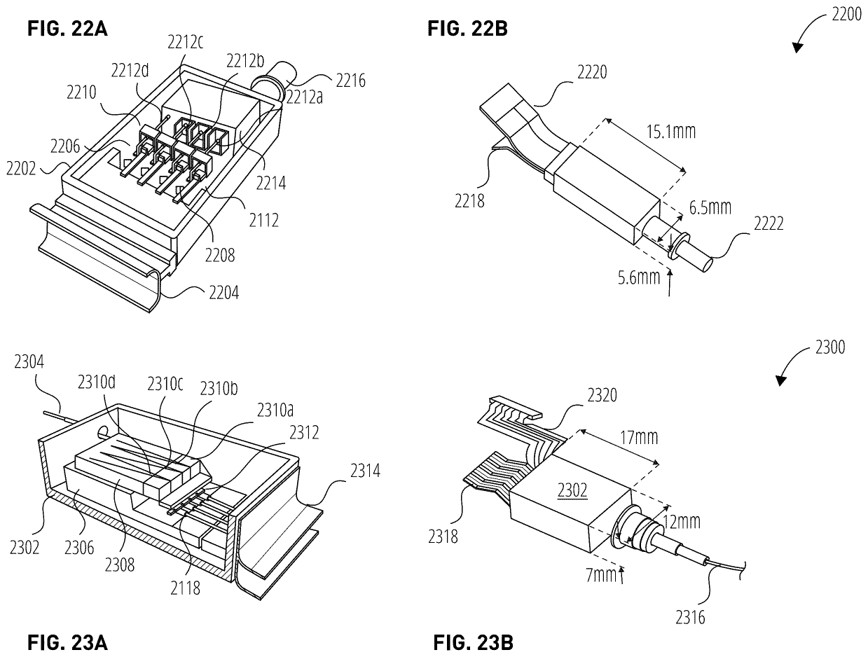

FIG. 21 illustrates a discrete beat node ammonia sensor 2100 in accordance with one embodiment. The discrete beat node ammonia sensor 2100 may be a 10 k version. The discrete beat node ammonia sensor 2100 may comprise a sensor module 2102, a passive sensor 2104, an active sensor 2106, a probe laser 2108, an excitation laser 2110, a thermo-electric cooler 2112, a probe photodetector 2114, an excitation photodetector 2116, a transimpedance amplifier 2118, a probe laser control 2120, an excitation laser control 2122, a temperature control 2124, a signal generator 2126, a lock-in amplifier 2128, an ADC 2130, a microcontroller unit 2132, a TOSA 2200, and a ROSA 2300.

The TOSA 2200 comprises a probe laser 2108, an excitation laser 2110, and a thermo-electric cooler 2112, as shown in FIG. 21. The TOSA 2200 may further comprise a transimpedance amplifier 2118, a package 2202, an electrical input interface 2204, an electro-absorption-modulated laser 2206, which may provide the probe laser 2108 and excitation laser 2110, a monitor photodetector 2208, a lens 2210, a lane 2212a, a lane 2212b, a lane 2212c, a lane 2212d, a built-in spatial multiplexer optics 2214, an optical output interface 2216, a flexible printed circuit for high-frequency signals 2218, a flexible printed circuit for direct current bias 2220, and a Lucent Connector or LC receptacle 2222.

The ROSA 2300 may include a probe photodetector 2114, an excitation photodetector 2116, and transimpedance amplifiers 2118 as shown in FIG. 21. The ROSA 2300 may further comprise a package 2302, an optical input interface 2304, a base 2306, a built-in spatial demultiplexer optics 2308, a lane 2310a, a lane 2310b, a lane 2310c, a lane 2310d, a photodetector array 2312 which may provide the probe photodetector 2114 and excitation photodetector 2116, an electrical output interface 2314, a fiber 2316, a flexible printed circuit for high-frequency signals 2318, and a flexible printed circuit for direct current bias 2320.

Reference may be made to the following publications:
KIM, Jae-Woo, et al., “Design and Implementation Scheme of QSFP28 Optical Transceiver for Long-Reach Transmission Using PAM4 Modulation,” Applied Sciences 11, 2803 (2021).
MOCHIZUKI, Keita, et al., “Assembly Technologies for Integrated Transmitter/Receiver Optical Sub-Assembly Modules,” IEICE Transactions on Electronics Vol. E100-C, No. 2 (2017): 187-195.
“QSFP-CWDM4-100G Datasheet,” FS. http://www.fs.com.

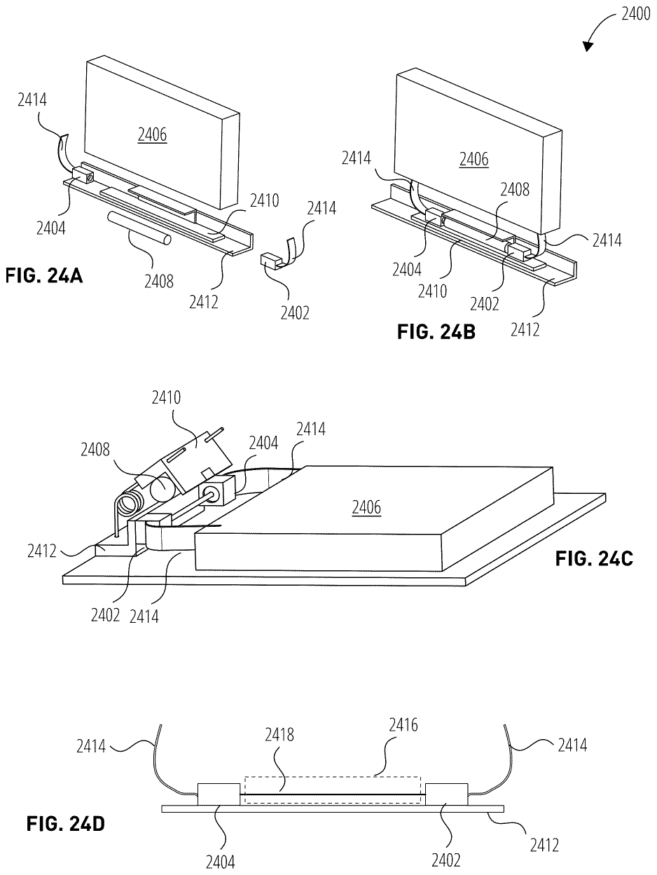

FIG. 24A-FIG. 24D illustrate a real-time calibration configuration with a reference cell 2400 in accordance with one embodiment. FIG. 24A provides an exploded view of included components. FIG. 24B and FIG. 24C provide isometric assembled views with the reference cell in differing positions. FIG. 24D provides a side elevation view.

The real-time calibration configuration with a reference cell 2400 may include a TOSA 2402 and ROSA 2404, sensor electronics 2406, a reference cell 2408, a heat sink 2410 to dissipate heat energy generated by the TOSA 2402 and ROSA 2404, a frame 2412 for mounting the TOSA 2402, ROSA 2404, and heat sink 2410 and supporting the reference cell 2408, and flexible printed circuit boards or cables 2414 connecting the TOSA 2402 and ROSA 2404 to the sensor electronics 2406.

FIG. 24B and FIG. 24D show the real-time calibration configuration with a reference cell 2400 in a position where the reference cell 2408 is positioned between the TOSA 2402 and ROSA 2404, within a gas detection area 2416 through which may pass the laser beams 2418 such as the probe laser and excitation laser described above. This is shown particularly in FIG. 24D.

FIG. 24C shows the real-time calibration configuration with a reference cell 2400 in an alternative position where the reference cell 2408 is elevated out of the gas detection area 2416. By configuring the gas reference cell 2408 such that it may be moved to incorporate the beam or have the beam outside of the cell, calibration may be performed in-situ, in real time.

FIG. 25A-FIG. 25G illustrate a photonic gas sensor with configurable add-ons 2500 in accordance with one embodiment. The photonic gas sensor with configurable add-ons 2500 may include electronics 2502 for control, signal processing, output connectivity, and other electronic functions as will be well understood by one of ordinary skill in the art, a TOSA 2504, a ROSA 2506, and glass windows 2508 to either side of an open area 2510 allowing passage of a reference beam 414, test beam 412, and excitation beam 408, as introduced with respect to FIG. 4, from the TOSA 2504 to the ROSA 2506, as shown in FIG. 25A, in order to test a gas or gases present within the open area 2510.

The electronics 2502 may include circuitry, mechanical elements, and software supporting the operation of the photonic gas sensor with configurable add-ons 2500. The TOSA 2504 may include laser diodes or other devices configured to generate the lasers needed, as well as collimating lenses, reflecting elements, optical isolators, and other features in support of the operation of the desired photonic gas sensor configuration, as disclosed herein. The glass windows 2508 may separate and protect the internal elements of the photonic gas sensor with configurable add-ons 2500 from the outside, ambient conditions and elements while allowing the laser beams to pass through without distortion or loss. The ROSA 2506 may include detectors such as photodiodes to detect the laser beams after they have passed through the sample under test, including a detector for the combined probe laser beams and the excitation laser or pump laser beam. Additional elements may be included for monitoring and data processing for the controlled operation of the photonic gas sensor with configurable add-ons 2500.

FIG. 25B illustrates a photonic gas sensor with configurable add-ons 2500 that further allows for an external pump or excitation beam. This embodiment includes a beam diverter 2512 that accepts an external single or multiple wavelength excitation laser 2514 and includes mirrors 2516 that reflect the single or multiple wavelength excitation laser 2514 through the beam diverter 2512 and out to an excitation laser photodiode 2518 while allowing the reference beam 414 and test beam 412 to pass from the TOSA 2504 to the ROSA 2506. The mirrors 2516 may be transparent to the wavelength of the test beam 412 and reference beam 414. The mirrors 2516 may include notch filtering properties for the reflection of the test beam 412, reference beam 414, and excitation beam 408 wavelengths. The mirror 2516 may be dichroic mirrors for combining and separating the single or multiple wavelength excitation laser 2514 and the test beam 412. In this manner, the photonic gas sensor with configurable add-ons 2500 may be adapted in the field to test additional gas compositions in the open area 2510. Dichroic materials may electively reflect beams of energy at different wavelengths in different percentage amounts based on their wavelengths.

FIG. 25C illustrates a photonic gas sensor with configurable add-ons 2500 configured with a gas reference cell 2520. In one embodiment, the gas reference cell 2520 may be repositionable within the open area 2510 of the photonic gas sensor with configurable add-ons 2500 similar to the reference cell 2408 of the real-time calibration configuration with a reference cell 2400 introduced above.

FIG. 25D illustrates a photonic gas sensor with configurable add-ons 2500 with a static gas reference cell 2520 which further includes a beam diverting element and actuator 2522 with mirrors 2524. The beam diverting element and actuator 2522 may be repositioned as shown in FIG. 25E, such that the mirrors 2524 redirect the test beam 412 and excitation beam 408 through the gas reference cell 2520 rather than the open area 2510.

FIG. 25F illustrates a photonic gas sensor with configurable add-ons 2500 which includes the gas reference cell 2520 and beam diverting element and actuator 2522 introduced in FIG. 25D. In this instance the photonic gas sensor with configurable add-ons 2500 further includes a disposable or cleanable insert 2526. The disposable or cleanable insert 2526 may include additional mirrors 2524. The disposable or cleanable insert 2526 may allow the introduction of a gas sample into the open area 2510 while preventing any damage or dirtying of the glass windows 2508 of the photonic gas sensor with configurable add-ons 2500. FIG. 25F illustrates the beam diverting element and actuator 2522 positioned to allow calibration using the gas reference cell 2520 while the disposable or cleanable insert 2526 is within the open area 2510.

Various embodiments may further improve upon the photonic gas sensor with configurable add-ons 2500 illustrated herein. One embodiment may incorporate one or more beams for heating a substant gas, fluid, or solid. One embodiment may implement synchronous detection using the probe laser signal without a signal having a wavelength for absorption, as is standard with wavelength modulation spectroscopy. In one embodiment, the photonic gas sensor with configurable add-ons 2500 may incorporate a multi-pass cell similar to those illustrated with respect to previous embodiments. In one embodiment, the photonic gas sensor with configurable add-ons 2500 may implement the ability to dynamically set an operating point. In one embodiment, offset compensation may be incorporated. In one embodiment, the photonic gas sensor with configurable add-ons 2500 may be configured with a wavelength tracker. In one embodiment, the photonic gas sensor with configurable add-ons 2500 may be configurable via a method for integrating the probe laser and excitation laser. In one embodiment, the photonic gas sensor with configurable add-ons 2500 may include elements for monitoring pollution of the optical windows. In one embodiment, the photonic gas sensor with configurable add-ons 2500 may include elements for cleaning the optical windows. In one embodiment, the photonic gas sensor with configurable add-ons 2500 may be configured with add-on modules that support a multibeam configuration, photonic integration, and electronic integration. In one embodiment, the photonic gas sensor with configurable add-ons 2500 may be configured with an add-on in support of multi-gas/multiwavelength measurement.

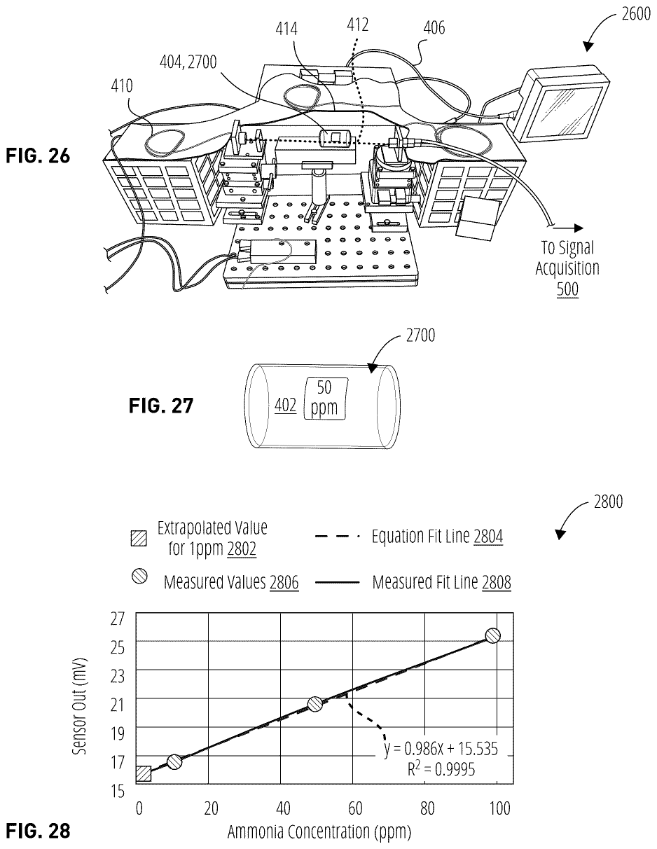

FIG. 26 illustrates an exemplary solution 2600 in accordance with one embodiment. The exemplary solution 2600 may include key elements of the photonic gas sensor 400 introduced in FIG. 4, including a sample reservoir 404, a pump laser or excitation laser 406 provided through an optical fiber cable, a probe laser 410, also provided through an optical fiber cable, and configured

The exemplary solution 2600 may in one embodiment include synchronous detection via a probe laser signal and not using a signal with wavelength for absorption. A test sample reservoir 2700, which may be used for the sample reservoir 404 previously introduced and which is described in greater detail below, is shown in place with the exemplary solution 2600 for reference.

FIG. 27 illustrates a test sample reservoir 2700 in accordance with one embodiment. The test sample reservoir 2700 may include a 50 ppm concentration of a gas such as ammonia gas.

FIG. 28 illustrates sensor output versus ammonia sample concentration data 2800 for one embodiment. Such data may be generated, for example, by the exemplary solution 2600 measuring the test sample reservoir 2700 in an experimental setting.

An extrapolated value 2802 is provided, along with an equation fit line 2804, which show in mathematical terms the results expected for accurate measurements by the exemplary solution 2600. As may be seen in the sensor output versus ammonia sample concentration data 2800, the measured values 2806 and measured fit line 2808 adhere closely to the results expected.

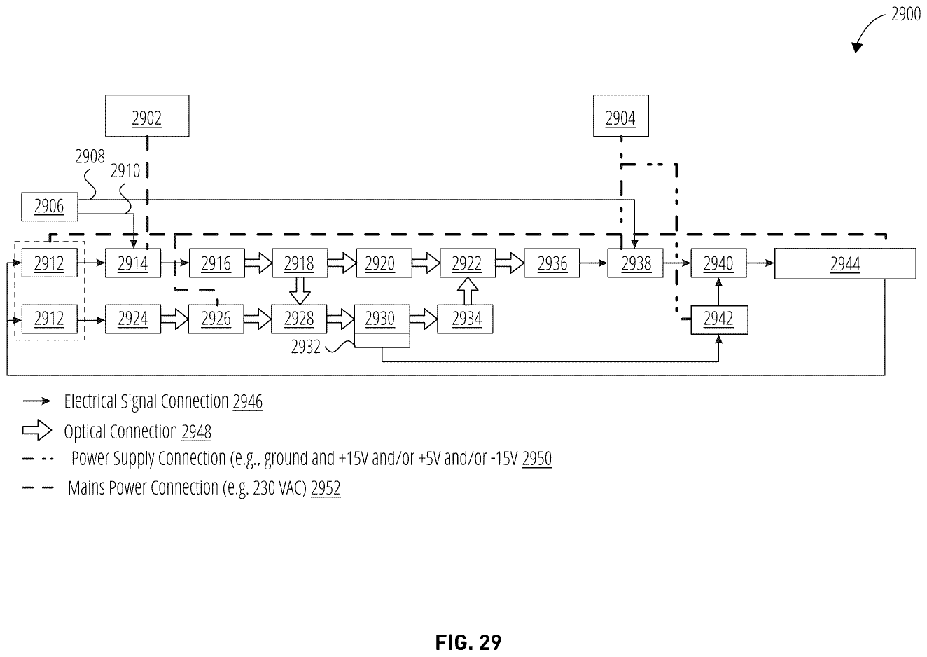

FIG. 29 illustrates an ammonia setup 2900 in accordance with one embodiment. The ammonia setup 2900 may involve synchronous detection via probe laser signal and not via signal with wavelength for absorption. The ammonia setup 2900 comprises a mains power 2902, a power supply unit 2904, an external function generator 2906, a sine wave 2908, a sine wave with offset 2910, a laser with thermoelectric cooling controller 2912, a modulator unit 2914, a 1,530 nm laser 2916, a 50/50 split 2918, a no ammonia 2920, a combine 2922, a 1,310 nm laser 2924, an erbium-doped fiber amplifier 2926, a combine 2928, an ammonia sample under test 2930, a negative temperature coefficient 2932, a 1,530 nm blocker 2934, a photodiode 2936, a lock-in amplifier 2938, an analog to digital converter 2940, a negative temperature coefficient circuit 2942, and a computing system 2944. These elements may be connected through electrical signal connections 2946, optical connections 2948, power supply connections 2950, and mains power connections 2952 as shown.

The ammonia setup 2900 may connect to mains power 2902 that is, for example, 230V alternating current from a building power source. The ammonia setup 2900 may additionally connect to a power supply unit 2904 which may be a lab power supply proving ground and positive and/or negative direct current power, such as +15V, +5V, −15V, etc. An external function generator 2906 may generate and provide sine wave 2908 and sine wave with offset 2910 electrical signals.

One or more lasers with thermoelectric cooling controllers 2912 may connect to a modulator unit 2914 and a 1,530 nm laser 2916 to provide a modulated excitation laser, and a 1,310 nm laser 2924 to provide a probe laser. The 1,310 nm laser 2924 may be amplified by an erbium-doped fiber amplifier 2926. The modulated 1,530 nm laser 2916 may be fed to a 50/50 split 2918 device, with one half of the resulting signal fed through a sample containing no ammonia 2920 and the other half fed to a combine 2928 element along with the output of the erbium-doped fiber amplifier 2926. The output of the combine 2928 element may be passed through an ammonia sample under test 2930 associated with a negative temperature coefficient 2932. The laser energy passed through the ammonia sample under test 2930 may be carried to a 1,530 nm blocker 2934, and the resulting output combined with the no ammonia 2920 output by a combine 2922 element.

The output of the combine 2922 stage may be passed to a lock-in amplifier 2938, the output of which may be input to an analog to digital converter 2940. The negative temperature coefficient 2932 may provide a signal to a negative temperature coefficient circuit 2942, the output of which may also be passed to the analog to digital converter 2940. Data from the analog to digital converter 2940 may be presented to a computing system 2944 for storage and analysis. In one embodiment, the computing system 2944 may be a miniature computer such as a Raspberry Pi. In one embodiment, the analog to digital converter 2940 may be provided as part of an electronics board such as an Arduino.

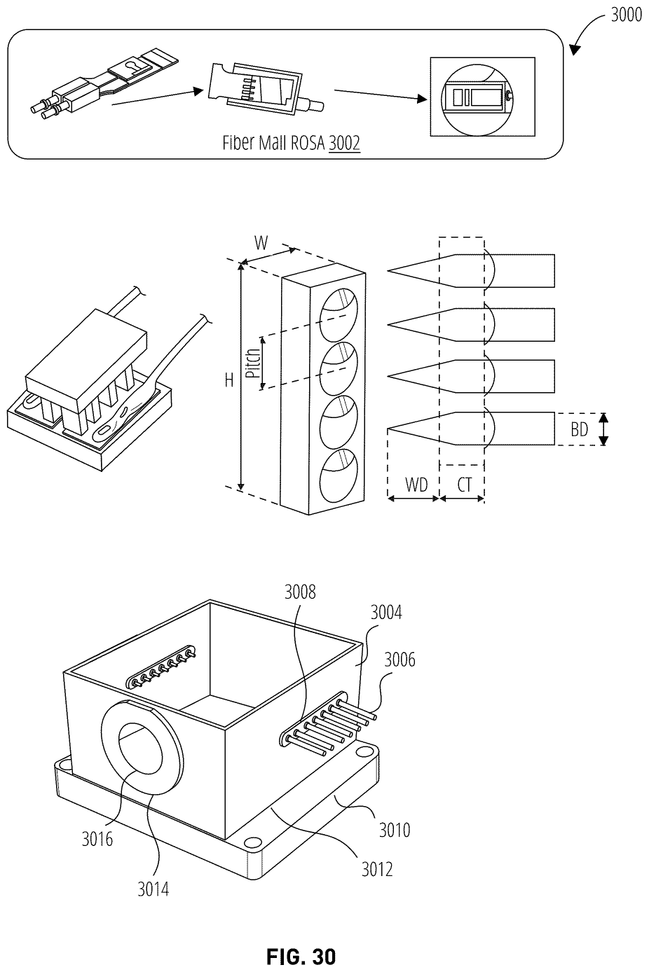

FIG. 30 illustrates an exemplary assembly integration 3000 in accordance with one embodiment. The exemplary assembly integration 3000 may include a fiber mall ROSA 3002, a package frame 3004, pins 3006, a feedthrough 3008, a base 3010, a frame buffer ring 3012, a lens holder 3014, and a window lens 3016.

The package frame 3004 may be made from Kovar. The pins 3006 may be coper-cored Kovar, ZrCu, or pure copper. The feedthrough 3008 for the pins 3006 may be ceramic using white Al₂O₃. The base 3010 may be tungsten copper. The frame buffer ring 3012 may be oxygen-free high thermal conductivity (OFHC) copper. The lens holder 3014 may be Kovar. The window lens 3016 may be sapphire anti-reflective (AR) coated.

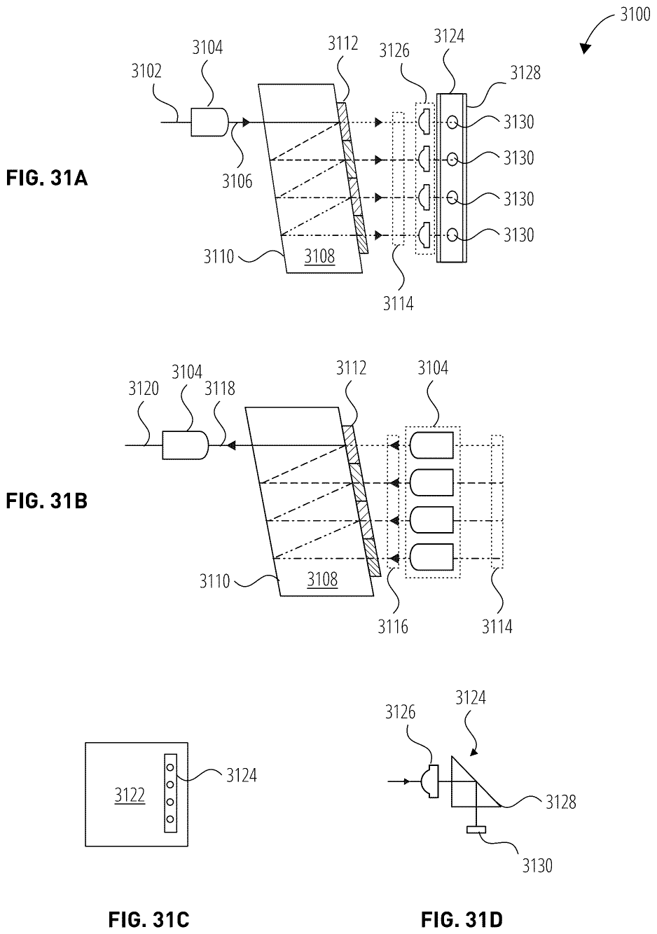

FIG. 31A-FIG. 31D illustrate aspects of a transceiver optics concept 3100 in accordance with embodiments as illustrated. The transceiver optics concept 3100 may include lasers 3102 and collimators 3104, as well as prisms 3108 configured with high-reflection coating 3110 and reflective filters 3112. FIG. 31A illustrates how a single laser 3102 beam may be passed through a collimator 3104 and the collimated beam 3106 traveling from thence to the prism 3108, where it may be selectively reflected and filtered into four separate laser 3102 beams, as shown. These beams may pass through micro-lenses 3126 to a photodetector chip 3124 which may include a prism 3128 to direct the beams onto photodetectors 3130. FIG. 31C and FIG. 31D provide more detailed illustrations of these aspects, including the printed circuit board 3122 which may support this transceiver optics concept 3100. The collimators 3104 may produce collimated beams which may be capable of providing a longer distance of overlap between a probe laser and excitation laser beams. This may result in more efficient heating of the gas sample under test as well as more efficient probing of the sample.

FIG. 31B illustrates an embodiment of the transceiver optics concept 3100 in which a similar configuration is used in reverse. Separated beams 3114 may be passed through collimators 3104 to form collimated separated beams 3116, which the high-reflection coating 3110 and reflective filters 3112 of the prism 3108 may act to combine into a combined beam 3118. The combined beam 3118 may pass through another collimator 3104, producing a collimated combined beam 3120.

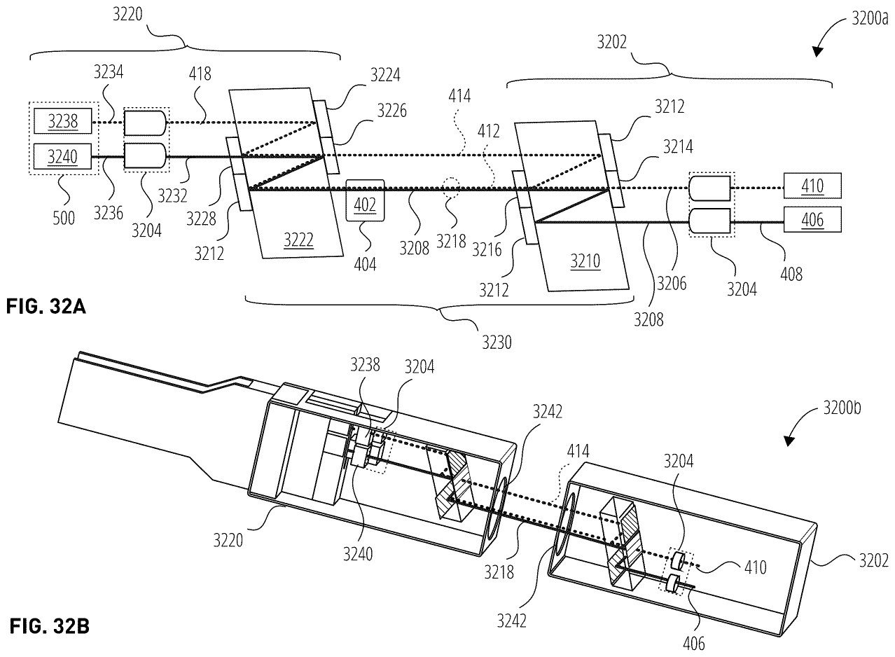

FIG. 32A illustrates an ammonia sensor optics concept 3200a in accordance with one embodiment. FIG. 32B illustrates an ammonia sensor with a packaged MZI 3200b in accordance with one embodiment. The ammonia sensor with a packaged MZI 3200b may be configured to implement the ammonia sensor optics concept 3200a or may be configured similarly but with additional or alternative features to implement the similar concepts illustrated herein.

Elements of the ammonia sensor optics concept 3200a may embody the gas sample under test 402, sample reservoir 404, excitation laser 406, excitation beam 408, probe laser 410, test beam 412, reference beam 414, and recombined signal 418 introduced in FIG. 4, as well as some portion of the signal acquisition 500 elements described with respect to FIG. 5. In another embodiment, similar elements may be used to configure the ammonia sensor optics concept 3200a with a Jamin interferometer The ammonia sensor optics concept 3200a may further include a TOSA 3202, collimating lenses 3204 a collimated probe beam 3206, a collimated excitation beam 3208, a prism 3210 with high-reflection coating 3212 and reflective filters 3214-3216, a combined beam 3218, a ROSA 3220, a prism 3222 with high-reflection coating 3212 and reflective filters 3224-3228, an output excitation beam 3232, a collimated recombined signal 3234, a collimated recombined signal 3234, a collimated output excitation beam 3236, a probe photodetector 3238 and an excitation photodetector 3240.

The TOSA 3202 may comprise the probe laser 410 having a 1,310 nm wavelength and an excitation laser 406 having a 1,530 nm wavelength. As will be readily understood by those of ordinary skill in the art, other laser wavelengths may be used, and the configuration illustrated adapted for these values, in order to test gases other than ammonia. The probe laser 410 and excitation laser 406 may send beams through collimating lenses 3204 to create the collimated probe beam 3206 and collimated excitation beam 3208. The TOSA 3202 may further include the prism 3210 configured with high-reflection coating 3212, reflective filter 3214, and reflective filter 3216, as shown. The high-reflection coating 3212 may reflect light of any wavelength incident upon it. The reflective filters shown here an in subsequent figures may be made from dichroic materials having properties such that they reflect beams of energy at different wavelengths in different manners based on their wavelengths. While filters are shown having desired properties for 1,530 nm and 1,310 nm beams, other materials may be used to accommodate lasers of other desirable wavelengths.

Reflective filter 3214 may selectively reflect 100% of the 1,530 nm energy from the collimated excitation beam 3208 but 0% of the 1,310 nm energy from the collimated probe beam 3206. Reflective filter 3216 may selectively reflect 0% of the 1,530 nm energy and 50% of the 1,310 nm energy, such that half of the latter might exit the prism 3210 and half be reflected back, then reflected forward by the high-reflection coating 3212, as shown. Thus, a reference beam 414 and test beam 412 may be formed from splitting the collimated probe beam 3206. Similarly, as shown, a combined beam 3218 may be formed from the test beam 412 and collimated excitation beam 3208 through the action of the high-reflection coating 3212, reflective filter 3214, and reflective filter 3216. The reference beam 414 and combined beam 3218 may then be projected from the TOSA 3202. The combined beam 3218 may pass through the sample reservoir 404 containing the gas sample under test 402. The reference beam 414 and combined beam 3218 may then arrive at the ROSA 3220.

The ROSA 3220 may comprise a prism 3222 configured with high-reflection coating 3212, reflective filter 3224, reflective filter 3226, and reflective filter 3228, as well as a probe photodetector 3238 and an excitation photodetector 3240, as shown. Reflective filter 3224 may reflect 0% of 1,530 nm energy and 100% of 1,310 nm energy (or in one embodiment may be replaced with high-reflection coating 3212). Reflective filter 3226 may reflect either 0% or 100% of 1,530 nm energy and 0% of 1,310 nm energy. Reflective filter 3228 may selectively reflect 0% of the 1,530 nm energy and 100% of the 1,310 nm energy. In this manner, the prism 3222 so configured may separate the combined beam 3218 into the test beam 412 and an output excitation beam 3232 and recombine the test beam 412 and reference beam 414 into a recombined signal 418. The recombined signal 418 and output excitation beam 3232 may pass through collimating lenses 3204 to form the collimated recombined signal 3234 and collimated output excitation beam 3236, which may be received at the probe photodetector 3238 and excitation photodetector 3240 of the ROSA 3220 respectively, as shown.

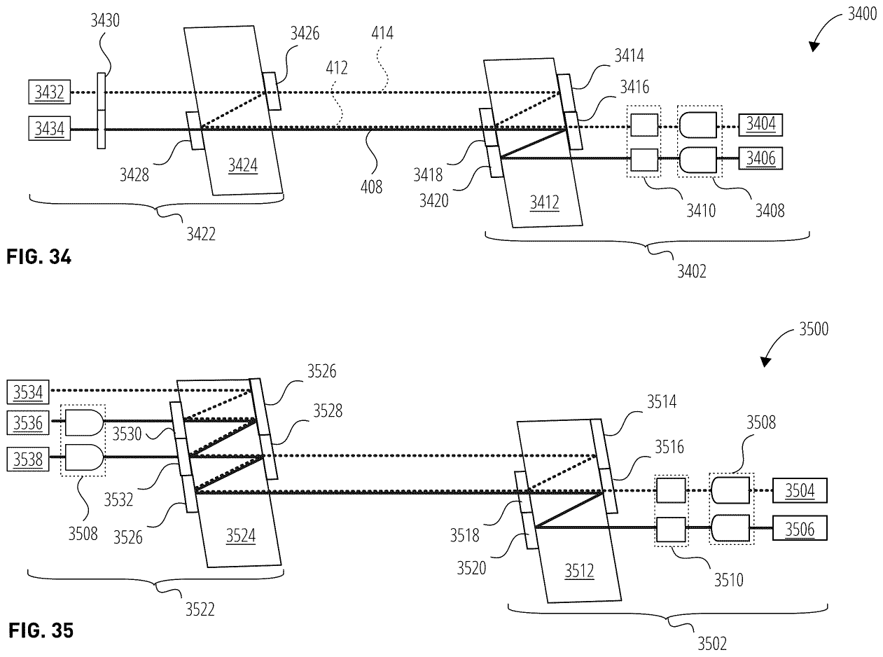

The prism 3210 of the TOSA 3202 and the prism 3222 of the ROSA 3220 may thus interact in this manner to form an interferometer 3230 to split and recombine the beams from the lasers of the TOSA 3302, such as the Mach-Zehnder interferometer 416 introduced in FIG. 4 or a Jamin interferometer if so configured. The reflective filters of the TOSA 3202 and ROSA 3220 may be adjusted as appropriate to the laser wavelengths used to adapt the disclosed design to additional gases. In one embodiment, the prisms may further incorporate anti-reflective coatings to improve the transmission of beams into their structures, those these coatings are not shown. The additional optics concepts shown in FIG. 34-FIG. 39 incorporate similar structures and rely upon similar concepts and are described below primarily in terms of their differing configurations.

FIG. 32B illustrates an ammonia sensor with a packaged MZI 3200b assembly integration in accordance with one embodiment. The assembly integration may be configured as the TOSA 3202 and ROSA 3220 introduced in FIG. 32A, which may each further incorporate optical windows 3242 that may in some embodiments be subject to pollution monitoring and cleaning.

Various methods for accommodation of pollution/biofouling may include forced surface airflow for separation of window/environmental air at excitation beam (max 1 mm diameter), continuous adjustment via pollution measurement, cleaning of window by heating, cleaning of widows by pull of foils, and cleaning of window with chemical agents.

Reference may be made to the following publications:
CATHERINE, “Four Optical Packaging Processes,” Fiber Mall, https://www.fibermall.com/blog/optical-packaging-processes.htm, accessed Apr. 11, 2024.
“CUI Devices Series CP07-M Datasheet,” CUI Devices. http://www.cuidevices.com.

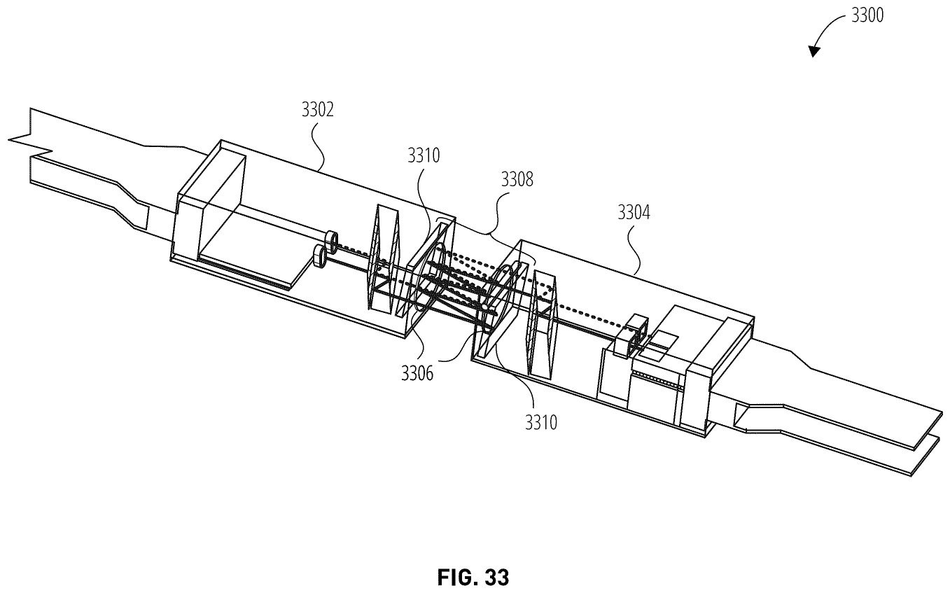

FIG. 33 illustrates an ammonia sensor optics concept with a multi-pass cell configuration 3300 in accordance with one embodiment. The ammonia sensor optics concept with a multi-pass cell configuration 3300 may resemble the ammonia sensor with a packaged MZI 3200b, including a TOSA 3302, a ROSA 3304, and optical windows 3306 similar to those shown for the ammonia sensor with a packaged MZI 3200b. The ammonia sensor optics concept with a multi-pass cell configuration 3300 may further include a multi-pass cell 3308, which may involve a multipath configuration in a hermetic environment.

In one embodiment, the multi-pass cell 3308 may include reflectors 3310 configured to reflect the beams back and forth a desired number of times to achieve a particular beam length within the distance between the TOSA 3302 and ROSA 3304. These reflectors 3310 may be configured as part of the package walls of the TOSA 3302 and ROSA 3304 while maintaining unreflective areas allowing passage of the TOSA 3302 exiting beams and ROSA 3304 entering beams.

Gas absorption is related to the length of the gas light interaction. By using reflectors 3310 that are aligned in such a way that the transmitted beam is reflected back and forth several times while shifting every cycle a small distance and ending the reflections at a point such that the last reflection enters the ROSA 3304 in a defined manner, a longer beam length may be achieved without enlarging the sensor device.

A number of methods may be used to accommodate pollution and biofouling of the optical window 3306 in harsh environments. These may include continuous adjustment via pollution measurement, cleaning of optical windows 3306 by heating, cleaning of optical windows 3306 by pull of foils, and cleaning of optical windows 3306 with chemical solutions.

FIG. 34 illustrates an ammonia sensor optics concept 3400 in accordance with one embodiment. Components similar to those described with respect to previous embodiments may perform similar functions herein.

The ammonia sensor optics concept 3400 may include a TOSA 3402 comprising a 1,310 nm probe distributed-feedback laser 3404, a 1,530 nm excitation distributed-feedback laser 3406, collimators 3408, optical isolators 3410, and a prism 3412. The probe distributed-feedback laser 3404 and excitation distributed-feedback laser 3406 beams may pass through the collimators 3408 and the optical isolators 3410 may direct the collimated beams to be incident upon the prism 3412 without crosstalk between the beams or other light energy contamination. The prism 3412 may include a reflective filter 3414, a reflective filter 3416, a reflective filter 3418, and a reflective filter 3420. The reflective filter 3414 may reflect 100% of 1,310 nm energy. The reflective filter 3416 may reflect 100% of 1,530 nm energy and 0% of 1,310 nm energy. The reflective filter 3418 may reflect 0% of 1,530 nm energy and 50% of 1,310 nm energy. The reflective filter 3420 may reflect 100% of 1,530 nm energy. In this manner, the TOSA 3402 prism 3412 may generate a test beam 412, a reference beam 414, and an excitation beam 408 that may be incident upon the ROSA 3422 of the ammonia sensor optics concept 3400 as shown.

The ROSA 3422 of the ammonia sensor optics concept 3400 may include a prism 3424 configured with a reflective filter 3426, a reflective filter 3428, optical isolators 3430, a probe photodetector 3432, and an excitation photodetector 3434. The reflective filter 3426 may reflect 50% of 1,310 nm energy and 0% of 1,530 nm energy. The reflective filter 3428 may reflect 100% of 1,310 nm energy and 0% of 1,530 nm energy. In this manner, the energy from the test beam 412, reference beam 414, and optical isolator 3430 may pass through optical isolators 3430 onto the probe photodetector 3432 and excitation photodetector 3434 as shown.

FIG. 35 illustrates an ammonia sensor optics concept 3500 in accordance with one embodiment. Detailed layout for the probe and excitation laser beams may be applied to an ammonia sensor optics concept. Components similar to those described with respect to previous embodiments may perform similar functions herein.

The ammonia sensor optics concept 3500 may include a TOSA 3502 that comprises a probe distributed-feedback laser 3504, an excitation distributed-feedback laser 3506, collimators 3508, optical isolators 3510, and a prism 3512. The prism 3512 may be configured with a reflective filter 3214, a reflective filter 3516, a reflective filter 3518, and a reflective filter 3520. The reflective filter 3514 may reflect 100% of 1,310 nm energy. The reflective filter 3516 may reflect 100% of 1,530 nm energy and 0% of 1,310 nm energy. The reflective filter 3518 may reflect 0% of 1,530 nm energy and 50% of 1,310 nm energy. The reflective filter 3520 may reflect 100% of 1,530 nm energy. In this manner, the TOSA 3502 may transmit laser beams to the ROSA 3522 as shown.

The ROSA 3522 may comprise a prism 3524 configured with high-reflection coating 3526, a reflective filter 3528, a reflective filter 3530, and a reflective filter 3532, as well as a probe photodetector 3534, an excitation photodetector 3536, and an excitation photodetector 3538 configured to receive the laser energy as shown. The reflective filter 3528 may reflect 50% of 1,310 nm energy and 100% of 1,530 nm energy. The reflective filter 3530 may reflect 100% of 1,310 nm energy and 0% of 1,530 nm energy. The reflective filter 3532 may reflect 100% of 1,530 nm energy and 50% of 1,310 nm energy. In this manner, the beams from the TOSA 3502 may be received at the probe photodetector 3534, excitation photodetector 3536, and excitation photodetector 3538 as shown. The excitation photodetector 3536 and excitation photodetector 3538 may receive the beams after they additionally pass through collimators 3508.

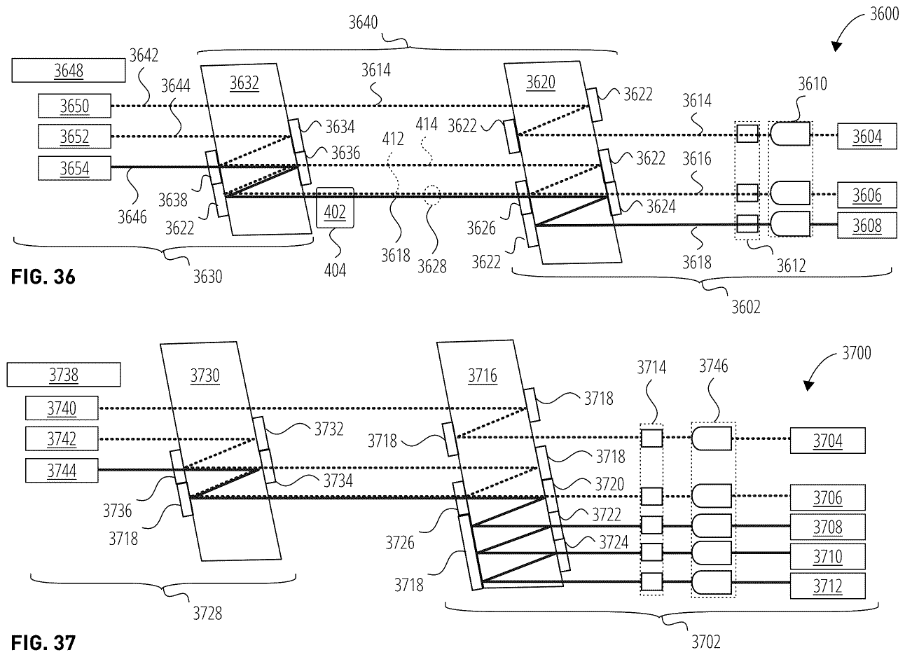

FIG. 36 illustrates an ammonia sensor optics concept with offset compensation 3600 in accordance with one embodiment. The ammonia sensor optics concept with offset compensation 3600 may involve splitting of probe laser signal and be configured as a differential amplifier. The ammonia sensor optics concept with offset compensation 3600 may include similar elements to the ammonia sensor optics concept 3200a illustrated in FIG. 32A, which may perform similar functions. However, these elements may be configured as shown for additional utility and may use beat node detection as introduced with respect to FIG. 9.

The ammonia sensor optics concept with offset compensation 3600 may include a TOSA 3602 comprising a 1,310 nm offset laser 3604, a 1,310 nm probe laser 3606, a 1,530 nm excitation laser 3608, collimating lenses 3610, optical isolators 3612, and a prism 3620. The offset laser 3604, probe laser 3606, and excitation laser 3608, may each pass through collimating lenses 3610 and optical isolators 3612 to form collimated offset beam 3614, collimated probe beam 3616, and collimated excitation beam 3618 before encountering the prism 3620. The prism 3620 may be configured with high-reflection coating 3622, reflective filter 3624, and reflective filter 3626. The reflective filter 3624 may reflect 100% of the 1,530 nm energy and 0% of the 1,310 nm energy. The reflective filter 3626 may reflect 0% of the 1,530 nm energy and 50% of the 1,310 nm energy.

In this manner, the collimated offset beam 3614 may pass out of the prism 3620, the collimated probe beam 3616 may be split into a test beam 412 and reference beam 414, and the test beam 412 may be combined with the collimated excitation beam 3618 to form a combined beam 3628 as shown. This configuration may result in the beams leaving the TOSA 3602 as shown, with the combined beam 3628 passing through a gas sample under test 402 in a sample reservoir 404, and the beams all then received at the ROSA 3630.

The ROSA 3630 may include a prism 3632 configured with high-reflection coating 3622, a reflective filter 3634, a reflective filter 3636, and a reflective filter 3638, as well as a beat node detector 3648, which may include or be coupled to a compensation photodetector 3650, a signal photodetector 3652, and a pollution photodetector 3654. The reflective filter 3634 may reflect 0% of 1,530 nm energy and 100% of 1,310 nm energy. The reflective filter 3636 may reflect 0 or 100% of 1,530 nm energy and 50% of 1,310 nm energy. The reflective filter 3638 may reflect 0% of 1,530 nm energy and 100% of 1,310 nm energy.

In this manner, the prism 3620 of the TOSA 3602 and the prism 3632 of the ROSA 3630 may interact as an interferometer 3640. The collimated offset beam 3614 may pass through the prism 3632 to become the output offset beam 3642 received at the compensation photodetector 3650. The combined beam 3628 may be split into the output excitation beam 3646 and the test beam 412. The test beam 412 and reference beam 414 may be recombined to form a recombined signal 3644 which may be received at the signal photodetector 3652. The output excitation beam 3646 may be received at the pollution photodetector 3654 and may aid in the detection of pollution.

FIG. 37 illustrates a multi-gas sensor optics concept with offset compensation 3700 in accordance with one embodiment. The multi-gas sensor optics concept with offset compensation 3700 may involve splitting of probe laser signals and be configured with a differential amplifier. Components similar to those described with respect to previous embodiments may perform similar functions herein.

The multi-gas sensor optics concept with offset compensation 3700 may comprise a TOSA 3702 that includes a 1,310 nm probe laser 3704, a 1,310 nm probe laser 3706, a 1,530 nm excitation laser 3708, a 1,530+x nm excitation laser 3710, and a 1,530+2x nm excitation laser 3712. The beams from lasers 3704-3712 may pass through collimators 3746 and optical isolators 3714 to be incident upon the prism 3716 of the TOSA 3702. The prism 3716 may be configured with high-reflection coating 3718, reflective filter 3720, reflective filter 3722, reflective filter 3724, and reflective filter 3726 as shown. The reflective filter 3720 may reflect 100% of 1,530 nm energy and 0% of 1,310 nm energy. The reflective filter 3722 may reflect 0% of 1,530 nm energy, 100% of 1,530+x nm energy, and 100% of 1,530+2x nm energy. The reflective filter 3724 may reflect 0% of 1,530+x nm energy and 100% of 1,530+2x nm energy. The reflective filter 3726 may reflect 0% of 1,530 nm energy and 50% of 1,310 nm energy.

Beams from the TOSA 3702 may be received by the ROSA 3728, comprising a prism 3730 configured with high-reflection coating 3718, reflective filter 3732, reflective filter 3734, and reflective filter 3736, as well as a beat node detector 3738, a compensation photodetector 3740, a signal photodetector 3742, and a pollution photodetector 3744. The reflective filter 3732 may reflect 0% of 1,530 nm energy and 100% of 1,310 nm energy. The reflective filter 3734 may reflect 0 or 100% of 1,530 nm energy and 50% of 1,310 nm energy. The reflective filter 3736 may reflect 0% of 1,530 nm energy and 100% of 1,310 nm energy.

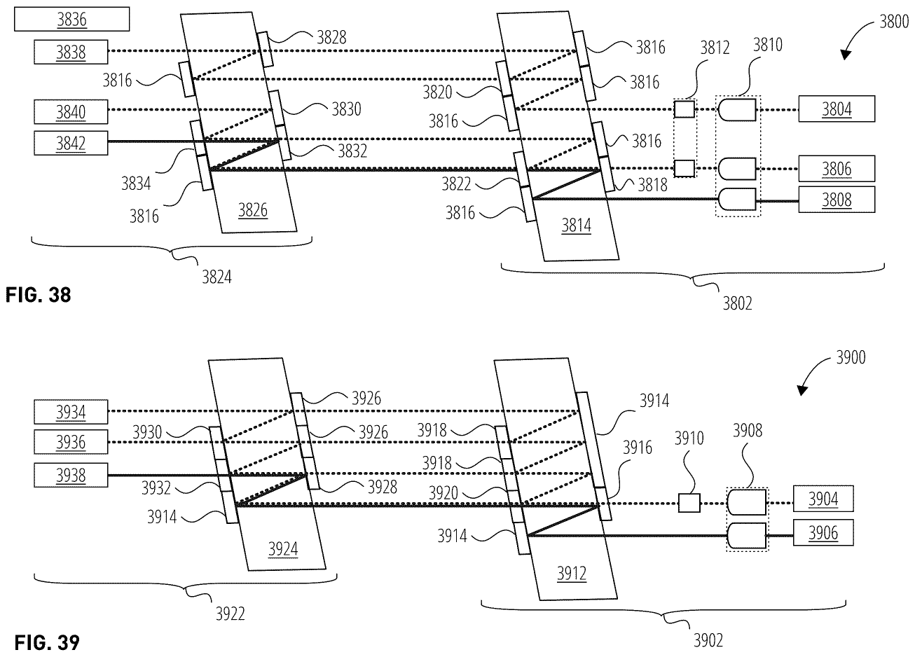

FIG. 38 illustrates an ammonia sensor optics concept with compensation of external environment influences 3800 in accordance with one embodiment. The ammonia sensor optics concept with compensation of external environment influences 3800 may be configured with a reference MZI with two lasers. Components similar to those described with respect to previous embodiments may perform similar functions herein.

The ammonia sensor optics concept with compensation of external environment influences 3800 may comprise a TOSA 3802 that includes a 1,310 nm probe laser 3804, a 1,310 nm probe laser 3806, a 1,530 nm excitation laser 3808, collimators 3810, optical isolators 3812, and a prism 3814. The beams from the lasers may pass through the collimators 3810, and beams from probe laser 3804 and probe laser 3806 may pass through optical isolators 3812, all beams then being incident upon the prism 3814 as shown. The prism 3814 may be configured with high-reflection coating 3816, reflective filter 3818, reflective filter 3820, and reflective filter 3822. The reflective filter 3818 may reflect 100% of 1,530 nm energy and 0% of 1,310 nm energy. Reflective filter 3820 may reflect 50% of 1,310 nm energy. Reflective filter 3822 may reflect 0% of 1,530 nm energy and 50% of 1,310 nm energy. The beams so generate may travel to the ROSA 3824 of the ammonia sensor optics concept with compensation of external environment influences 3800.

The ROSA 3824 may include a prism 3826 configured with high-reflection coating 3816, a reflective filter 3828, a reflective filter 3830, a reflective filter 3832, and a reflective filter 3834. The ROSA 3824 may also include a beat node detector 3836, a compensation photodetector 3838, a signal photodetector 3840, and a pollution photodetector 3842. The reflective filter 3828 may reflect 50% of 1,310 nm energy. The reflective filter 3830 may reflect 0% of 1,530 nm energy and 100% 1,310 nm energy. The reflective filter 3832 may reflect 0% or 100% of 1,530 nm energy and 50% of 1,310 nm energy. The reflective filter 3834 may reflect 0% of 1,530 nm energy and 100% of 1,310 nm energy. The laser beams received at the ROSA 3824 may thus be detected at the compensation photodetector 3838, signal photodetector 3840, and pollution photodetector 3842 as illustrated.

FIG. 39 illustrates an ammonia sensor optics concept with compensation for external environment influences 3900 in accordance with one embodiment. The ammonia sensor optics concept with compensation for external environment influences 3900 may be configured with a reference MZI with a laser that is the same as the probe laser. Components similar to those described with respect to previous embodiments may perform similar functions herein.

The ammonia sensor optics concept with compensation for external environment influences 3900 may include a TOSA 3902 with a 1,310 nm probe laser 3904 and a 1,530 nm excitation laser 3906. The probe laser 3904 may be passed through a collimator 3908 and an optical isolator 3910. The excitation laser 3906 may pass through a collimator 3908. The beams from these lasers may then be incident upon a prism 3912 of the TOSA 3902 configured with high-reflection coating 3914, a reflective filter 3916, a reflective filter 3918, and a reflective filter 3920. The reflective filter 3916 may reflect 100% of 1,530 nm energy and 0% of 1,310 nm energy. The reflective filter 3918 may reflect 50% of 1,310 nm energy. The reflective filter 3920 may reflect 0% of 1,530 nm energy and 50% of 1,310 nm energy. The resulting beams from the TOSA 3902 may pass from there to the ROSA 3922 of the ammonia sensor optics concept with compensation for external environment influences 3900 as shown.

The ROSA 3922 may include a prism 3924 configured with high-reflection coating 3914, a reflective filter 3926, a reflective filter 3928, a reflective filter 3930, and a reflective filter 3932. The ROSA 3922 may further include a compensation photodetector 3934, a signal photodetector 3936, and a pollution photodetector 3938. The reflective filter 3926 may reflect 50% of 1,310 nm energy. The reflective filter 3928 may reflect 0 or 100% of 1,530 nm energy and 50% of 1,310 nm energy. The reflective filter 3930 may reflect 50% of 1,310 nm energy. The reflective filter 3932 may reflect 0% of 1,530 nm energy and 100% of 1,310 nm energy. In this manner, the beams incident upon the prism 3924 of the ROSA 3922 may be transmitted to the compensation photodetector 3934, signal photodetector 3936, and pollution photodetector 3938 as shown.

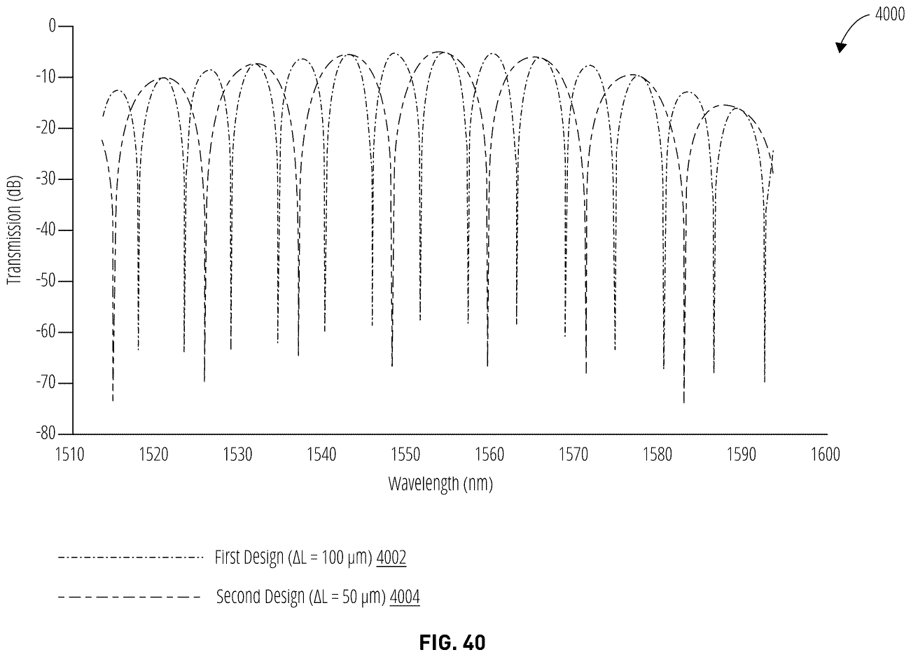

FIG. 40 illustrates a transmission spectrum of an imbalanced MZI 4000 in accordance with one embodiment. The spectrum may be achieved using different path lengths. The MZI may be used as a filter. Transmission strength in decibels (dB) is shown as a function of wavelength across the transmission spectrum for a first design 4002 and a second design 4004.

The group index n_{g} is used to determine the free-spectral range (FSR), which may be visualized as the shift in effective index versus wavelength (i.e., the spacing between adjacent peaks, ad described in Equation 1 below.

$${n}_{g} ( λ ) = {n}_{eff} ( λ ) - λ \cdot ( \frac{d}{d λ} {n}_{eff} ( λ ) ) \qquad Equation 1$$

The FSR for an MZI is given by Equation 2 below.

$$FSR = \frac{{λ}^{2}}{Δ L \cdot {n}_{g} ( λ )} \qquad Equation 2$$

The FSR thus depends on the imbalance length ΔL and the group index n_{g} of the waveguide. The first design 4002 for which the transmission spectrum of an imbalanced MZI 4000 is provided has a ΔL equal to 100 μm. The second design 4004 has a ΔL of 50 μm.

Reference may be made to the following publications:
RIZAL, C. S. and NIRAULA, B., “Compact Si-based asymmetric MZI waveguide on SOI as a thermo-optical switch,” Optics Communications (2017).
SCHNEIDER, Louisa Catharina, “Design, Fabrication and Analysis of a Mach-Zehnder Interferometer,” Silicon Photonics Design, Fabrication and Data Analysis Course, NSERC SiEPIC Program (2017).

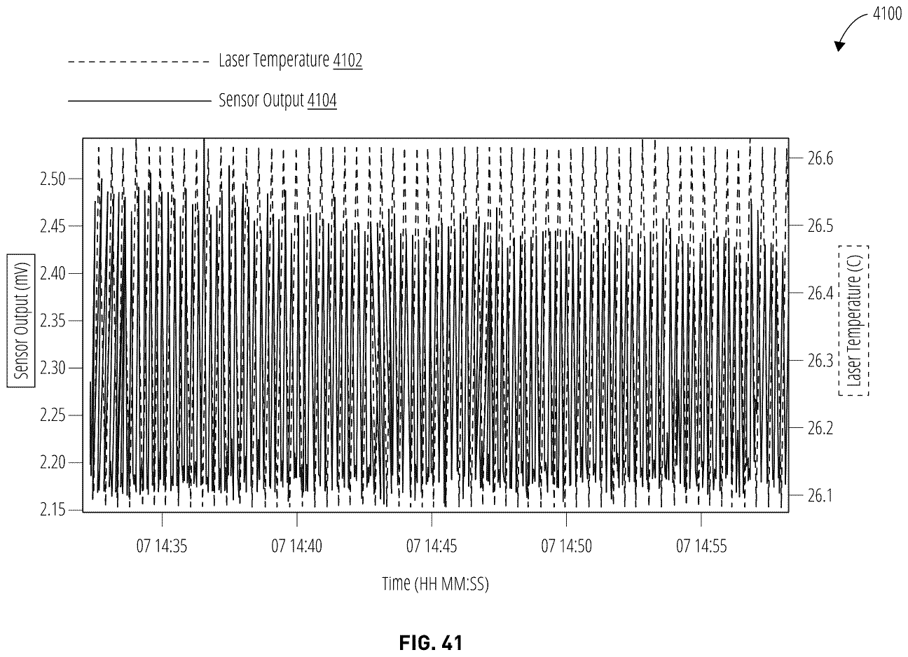

FIG. 41 illustrates a maximum lock-in amplifier output for ammonia at 10 ppm 4100 in accordance with one embodiment. The maximum lock-in amplifier output for ammonia at 10 ppm 4100 includes laser temperature 4102 and sensor output 4104 as a function of time as shown. Very high signals may be seen.

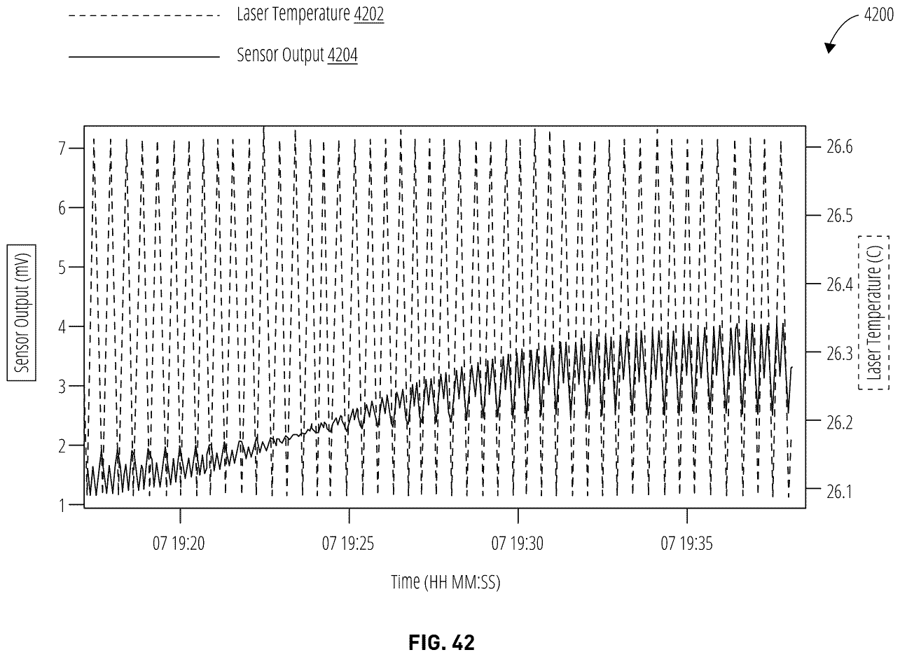

FIG. 42 illustrates a lock-in amplifier output for 100 ppm ammonia 4200 in accordance with one embodiment. The lock-in amplifier output for 100 ppm ammonia 4200 includes laser temperature 4202 and sensor output 4204 as a function of time as shown. Output levels may be dependent on the location of the background interference signal.

Additional graphs that measure over 1 hour may be useful in analyzing lock-in amplifier performance to show the following attributes:
Excitation laser time series (on and off state), which may show:
   Wavelength change (negative temperature coefficient or NTC value)
   Wavelength from an optical spectrum analyzer (OSA)
   Optical power (laser output and EDFA output)
   Power with lock-in probe modulation signal (laser output and EDFA output)
Probe laser time series, which may show:
   Wavelength change (NTC value)
   Wavelength from OSA
   Optical power (laser output)
   Power with lock-in probe modulation signal (laser output)

Reference may be made to the following resources:
https://www.epa.gov/aegl/agent-gb-sarin-results-aegl-program
https://www.techbriefs.com/component/content/article/35165-detecting-chemical-weapons-using-fourier-transform-infrared-spectroscopy
https://www.opticsjournal.net/Articles/OJ1c53ec25e4888d7b/Abstract
https://www.nature.com/articles/s41598-019-45945-y
https://pubs.rsc.org/en/content/articlelanding/2014/an/c4an00556b
chemical spectra, found in the University of Washington's VPL Molecular Spectra Search Engine (https://vplapps.astro.washington.edu/vplrangemicro.php).
The wavelength range in micro-meter units is between 0.1 μm and 100 μm, wavelength_low: 1.525 μm, wavelength_high: 1.675 μm.
Hydrogen sulfide (H₂S):

https://vpl.astro.washington.edu/spectra/h2spnnlimagesmicrons.htm
Methane (CH₄): https://vpl.astro.washington.edu/spectra/ch4pnnlimagesmicrons.htm
Ammonia (NH₃) https://vpl.astro.washington.edu/spectra/nh3pnnlimagesmicrons.htm
Benzene (C₆H₆) https://vpl.astro.washington.edu/spectra/c6h6pnnlimagesmicrons.htm
Ethene (C₂H₄): https://vpl.astro.washington.edu/spectra/c2h4pnnlimagesmicrons.htm
Ethanol (C₂H₅OH) https://vpl.astro.washington.edu/spectra/c2h5ohpnnlimagesmicrons.htm
Nitrous oxide (N₂O) https://vpl.astro.washington.edu/spectra/n2opnnlimagesmicrons.htm
Formaldehyde (H₂CO) https://vpl.astro.washington.edu/spectra/h2copnnlimagesmicrons.htm
N₂O, CH₄ and carbon dioxide (CO₂): https://hitran.org/lbl/#
H₂S, C₂H₂, NH₃: https://hitran.org/lbl/#
Water (H₂O), carbon monoxide (CO), ozone (O₃): https://hitran.org/lbl/#
Hydrogen iodide (HI), hydroxide (OH), hydrogen cyanide (HCN): https://hitran.org/lbl/#

The specific embodiments above are for explanatory purposes, as the disclosed solution may be reworked and achieved in a distinct but comparable manner evident to individuals skilled in the art without altering the essence of the disclosure. This disclosure on a photonic gas sensor may hold different applications in various areas such as numerous applications where gas sensing is needed.

References herein to words revised by language of estimation such as “about,” “substantially,” etc., are not to be limited to the exact value indicated.

References herein to words revised by language of estimation such as “vertical”, “horizontal,” “above,” etc., are created for instance of examples, and not to restrict, to ascertain a frame of reference. The terms “vertical” and “above” represent the term perpendicular to the “horizontal.”

Numerous embodiments of the current disclosure have been described for illustrative purposes but are not intended to be comprehensive or restricted to the embodiments unveiled. The choice of words employed in this work was selected to best explain the principles of the embodiments provided herein, the real-world application or technical advances over technologies found in the marketplace, and to allow others of ordinary skill in the art to understand the embodiments disclosed herein.

What is claimed is:

1. A method comprising:
exciting, using an excitation beam from an excitation laser, molecules of a gas sample under test in a sample reservoir;
applying, from a lock-in sine wave signal generator, a probe modulation signal to a probe laser;
splitting the probe laser output into a reference beam and a test beam;
passing the test beam through the sample reservoir;
recombing the test beam and the reference beam to create a recombined signal representing relative phase shift variations between the test beam and the reference beam;
detecting the recombined signal using a photodetector;
receiving, by a transimpedance amplifier, the detected recombined signal to generate a voltage signal carrying data from the photodetector;
receiving, by a lock-in amplifier, the voltage signal from the transimpedance amplifier and the probe modulation signal from the lock-in sine wave signal generator to generate a lock-in amplifier output signal comparing the voltage signal and the probe modulation signal;
converting, by an analog to digital converter, the lock-in amplifier output signal to a digital output signal;
processing, by a signal processing module, the digital output signal to determine gas sample concentration data.

2. The method of claim 1, wherein splitting the probe laser into the reference beam and the test beam is performed by a Mach-Zehnder interferometer or a Jamin interferometer.

3. The method of claim 1, wherein the gas sample under test includes ammonia.

4. The method of claim 1, further comprising:
passing the probe laser and the excitation laser through a transmitter optical sub-assembly (TOSA), to produce the reference beam, the test beam, and the excitation beam, wherein the TOSA includes TOSA collimating lenses, a TOSA prism, a TOSA high-reflection coating, and TOSA reflective filters;
passing the test beam and the excitation beam through the sample reservoir;
receiving the reference beam; and
receiving, after passing through the sample reservoir, the test beam and the excitation beam, through a receiver optical sub-assembly (ROSA), to produce the recombined signal, wherein the ROSA includes ROSA collimating lenses, a ROSA prism, a ROSA high-reflection coating, and ROSA reflective filters.

5. The method of claim 4, further comprising:
applying, with the TOSA, the probe laser and the excitation laser through the TOSA collimating lenses to produce a collimated probe beam and a collimated excitation beam; and
passing the collimated probe beam and the collimated excitation beam through the TOSA prism, resulting in the reference beam and the test beam formed from the collimated probe beam, and a combined beam formed from the test beam and the collimated excitation beam.

6. The method of claim 4, further comprising:
separating, with the ROSA prism, a combined beam into the test beam and an output excitation beam, wherein the combined beam is formed from the test beam and the recombining the test beam and the reference beam to create the recombined signal;
directing the output excitation beam and recombined signal through the ROSA collimating lenses to create a collimated output excitation beam and a collimated recombined signal; and
directing the collimated recombined signal to a probe photodetector and the collimated output excitation beam to an excitation photodetector.

7. The method of claim 4, wherein the TOSA reflective filters have properties such that they selectively reflect beams of energy at different wavelengths in different percentage amounts based on their wavelengths.

8. The method of claim 4, wherein the ROSA reflective filters have properties such that they selectively reflect beams of energy at different wavelengths in 0% or 100% amounts based on their wavelengths.

9. The method of claim 4, wherein the TOSA high-reflection coating reflects light, of any wavelength, incident upon it.

10. A method comprising:
applying, from a block signal generator, an excitation modulation signal to an excitation laser;
exciting, using an excitation beam from the excitation laser, molecules of a gas sample under test in a sample reservoir;
applying, from a lock-in sine wave signal generator, a probe modulation signal to a probe laser;
splitting the probe laser output into a reference beam and a test beam;
passing the test beam through the sample reservoir;
recombing the test beam and the reference beam to create a recombined signal representing relative phase shift variations between the test beam and the reference beam;
detecting the recombined signal using a photodetector;
receiving, by a transimpedance amplifier, the detected recombined signal to generate a voltage signal carrying data from the photodetector;
receiving, by a lock-in amplifier, the voltage signal from the transimpedance amplifier and the probe modulation signal from the lock-in sine wave signal generator to generate a lock-in amplifier output signal comparing the voltage signal and the probe modulation signal;
converting, by an analog to digital converter, the lock-in amplifier output signal to a digital output signal;
processing, by a signal processing module, the digital output signal to determine gas sample concentration data.

11. The method of claim 10, wherein splitting the probe laser into the reference beam and the test beam is performed by a Mach-Zehnder interferometer or a Jamin interferometer.

12. The method of claim 10, wherein the gas sample under test includes ammonia.

13. The method of claim 10, further comprising:
passing the probe laser and the excitation laser through a transmitter optical sub-assembly (TOSA), to produce the reference beam, the test beam, and the excitation beam, wherein the TOSA includes TOSA collimating lenses, a TOSA prism, a TOSA high-reflection coating, and TOSA reflective filters;
passing the test beam and the excitation beam through the sample reservoir;
receiving the reference beam; and
receiving, after passing through the sample reservoir, the test beam and the excitation beam, through a receiver optical sub-assembly (ROSA), to produce the recombined signal, wherein the ROSA includes ROSA collimating lenses, a ROSA prism, a ROSA high-reflection coating, and ROSA reflective filters.

14. The method of claim 13, further comprising:
applying, with the TOSA, the probe laser and the excitation laser through the TOSA collimating lenses to produce a collimated probe beam and a collimated excitation beam; and
passing the collimated probe beam and the collimated excitation beam through the TOSA prism, resulting in the reference beam and the test beam formed from the collimated probe beam, and a combined beam formed from the test beam and the collimated excitation beam.

15. The method of claim 13, further comprising:
separating, with the ROSA prism, a combined beam into the test beam and an output excitation beam, wherein the combined beam is formed from the test beam and the excitation beam;
recombining the test beam and the reference beam to create the recombined signal;
directing the output excitation beam and recombined signal through the ROSA collimating lenses to create a collimated output excitation beam and a collimated recombined signal; and
directing the collimated recombined signal to a probe photodetector and the collimated output excitation beam to an excitation photodetector.

16. The method of claim 13, wherein the TOSA reflective filters have properties such that they selectively reflect beams of energy at different wavelengths in different percentage amounts based on their wavelengths.

17. The method of claim 13, wherein the ROSA reflective filters have properties such that they selectively reflect beams of energy at different wavelengths in 0% or 100% amounts based on their wavelengths.

18. The method of claim 13, wherein the TOSA high-reflection coating reflects light, of any wavelength, incident upon it.

19. A method comprising:
exciting, using an excitation laser, molecules of a gas sample in a sample reservoir;
applying, from a lock-in sine wave signal generator, a probe modulation signal to a probe laser and a reference laser;
passing a test beam from the probe laser through the sample reservoir;
receiving, by a beat node detector, the test beam and output from the reference laser, into a photodetector, thereby creating a combined detected signal representing a difference in optical frequencies between the test beam and the output from the reference laser;
receiving, by a transimpedance amplifier, the combined detected signal to generate a voltage signal carrying data from the photodetector;
receiving, by a frequency modulation demodulator, the voltage signal from the transimpedance amplifier to produce a demodulated output signal;
receiving, by a lock-in amplifier, the demodulated output signal and the probe modulation signal from the lock-in sine wave signal generator to generate a lock-in amplifier output signal comparing the demodulated signal and the probe modulation signal;
converting, by and analog to digital converter, the lock-in amplifier output signal to a digital output signal;
processing, by a signal processing module, the digital output signal to determine gas sample concentration data.

20. The method of claim 19, wherein the gas in the sample reservoir is ammonia.
